(19) United States
(12) Patent Application Publication
(10) Pub. No.: US 2011/0034347 A1
(43) Pub. Date: Feb. 10, 2011

(54) Signatures Associated with Rejection or Recurrence of Cancer

(76) Inventors: Masoud Manjili, Richmond, VA (US); Maciej Kmieciak, Richmond, VA (US); Harry D. Bear, Richmond, VA (US); Catherine I. Dumur, Richmond, VA (US); Michael O. Idowu, Richmond, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/859,933

(22) Filed: Aug. 20, 2010

Related U.S. Application Data
(63) Continuation-in-part of application No. PCT/US2009/034676, filed on Feb. 20, 2009.
(60) Provisional application No. 61/030,613, filed on Feb. 22, 2008.

(51) Int. Cl. C40B 30/04; C12Q 1/68; C12M 1/34; C40B 40/06; C40B 60/12

(52) U.S. Cl. 506/9; 435/6; 435/287.2; 506/16; 506/39

(57) ABSTRACT

Methods and tools for assessing the prognosis for patients following treatment of primary tumors are provided. The methods involve identifying immune-related genetic markers whose differential expression patterns at tumor lesions are indicative of either tumor recurrence or recurrence-free survival. The methods and tools of the invention assist physicians by providing objective decision-making tools for planning patient treatment protocols.

### BACKGROUND OF THE INVENTION

1. Field of the Invention

The invention generally relates to assessing the prognosis for cancer. In particular, the invention provides methods for identifying immune-related genetic markers whose expression patterns at tumor lesions are indicative of patient prognosis following treatment of the primary tumor.

2. Background of the Invention

Challenges in the immune therapy of cancers include a limited understanding of the requirements for tumor rejection and prevention of recurrences after successful therapy. Evaluation of T cell responses in human tumors, based predominantly on the metastatic melanoma model have clearly shown that the tumor bearing status primes systemic immune responses against tumor-associated antigens, which, however, are insufficient to induce tumor rejection (1, 2). Moreover, the experience gathered through the induction of tumor antigen-specific T cells by vaccines has shown that the presence of tumor antigen-specific T cells in the circulation (3, 4) or in the tumor microenvironment (5, 6) does not directly correlate with successful rejection or prevention of recurrence (7). Similarly, patients with pre-existing immune responses against HER-2/neu are not protected from the development of HER-2/neu expressing breast cancers (8). Although several and contrasting reasons have been proposed to explain this paradox, two lines of thoughts summarize these hypotheses; either tolerogenic and/or immune suppressive properties of tumors may hamper T cell function (9-11) or characteristics of the tumor microenvironment could induce tumor escape and evade the anti-tumor function of effector T cells (12, 13).

In spite of this paradoxical coexistence of tumor specific T cells and their target antigen-bearing cancer cells, recent observations in cancer patients suggest that T cells control tumor growth and mediate its rejection. Galon J et al. and others (14-16) observed that T cells modulate the growth of human colon cancer and T cell infiltration of primary lesions may forecast a better prognosis. In addition, these authors observed that tumor-infiltrating T cells in cancers with good prognosis displayed transcriptional signatures typical of activated T cells such as the expression of interferon stimulated genes (ISGs), IFN-γ itself and cytotoxic molecules, in particular granzyme-B (15). Similar observations were reported by others in human ovarian carcinoma (17). Spontaneous regression of melanoma has also been reported to be mediated by complex and systemic immune responses rather than by T cell or antibody responses alone (18). These important observations derived from human tissues provide novel prognostic markers but cannot address the causality of the association between T cell infiltration and natural history of cancer. Recent reports based on adoptive transfer of tumor-specific T cells suggest a cause-effect relationship between the administration of T cells and tumor rejection (19). However, the complexity of the therapy associated with adoptive transfer of T cells which includes immune ablation and systemic administration of IL-2 prevents a clear interpretation of this causality.

The prior art has thus far failed to provide reliable methods to characterize successful and unsuccessful immune system responses to the presence of a tumor, and to accurately establish a prognosis prior to or following treatment of primary tumors. This is important because an accurate prognosis is extremely valuable in assessing treatment options for the patient.

### SUMMARY OF THE INVENTION

The present invention introduces a method for the analysis of tumor microenvironments in order to assess the prognosis of patients with carcinomas. The invention is based on the identification of immune-related genetic markers whose expression patterns at tumor lesions can be used to predict whether or not the patient is mounting effective immune response against the tumor that is likely to reject residual or recurring tumors, or not. In other words, the pattern of gene expression is indicative of the likelihood of relapse or recurrence of the cancer after treatment. 299 genes have been identified which, when upregulated, are associated with a low risk of relapse, i.e. with a high probability of a relapse-free recovery (see Table 4). 50 genes have been identified which, when upregulated, are associated with a high risk of relapse, i.e. with a high probability of recurrence of the cancer. The ability to identify these patterns of gene expression permit health practitioners to optimize treatment protocols for cancer patients. For example, a patient that is categorized as unlikely to experience a relapse need not be subjected to aggressive measures which can be traumatic and debilitating in and of themselves. On the other hand, patients identified as likely to experience a recurrence of the cancer can be aggressively treated in order to provide the best possible chance of long-term survival. The invention therefore provides an objective decision-making tool for physicians regarding how to treat patients with, for example, ductal carcinoma in situ (DCIS) or other invasive carcinomas. The invention also provides kits containing ready-to-use microarray chips, two tier computer software data analysis and statistical methods for determining efficacious treatment options commensurate with the prognoses that are provided.

The invention provides a method for determining a probability of relapse of a patient with a tumor. The method comprises the steps of 1) obtaining a sample of the tumor; 2) measuring expression levels of immune system genes associated with the tumor; 3) determining, using the expression levels of the immune system genes, a pattern of expression of immune system genes associated with the tumor; and, based on the pattern of expression, 4) assigning the patient to a group selected from the group consisting of: i) patients who are likely to experience a relapse; and ii) patients who are unlikely to experience a relapse. In some embodiments, the immune system genes include at least two genes listed in Tables 4 and 5. In other embodiments, the immune system genes include at least one gene listed in Table 4 and at least one gene listed in Table 5. In yet other embodiments, the immune system genes include all genes listed in Table 4 and Table 5. The immune system genes may be from one or more pathways such as B cell development, antigen presentation, graft vs host disease signaling, interferon signaling and primary immunodeficiency signaling. In one embodiment, the pattern of expression of immune system genes shows upregulation of one or more genes listed in Table 4 and downregulation of one or more genes listed in Table 5, and the patient is assigned to the group of patients who are unlikely to experience a relapse. In another embodiment, the pattern of expression of immune system genes shows upregulation of one or more genes listed in Table 5 and downregulation of one or more genes listed in Table 4, and the patient is assigned to said group of patients who are likely to experience a relapse. In some embodiments, the tumor is a breast cancer tumor.

The invention further provides a method for developing a treatment protocol for a patient with a tumor. The method comprises the steps of: 1) obtaining a sample of the tumor; 2) determining a pattern of expression of immune system genes in the tumor; then, based on the pattern of expression of immune system genes, 3) categorizing the patient as a patient that is likely to undergo a relapse or as a patient that is unlikely to undergo a relapse; and 4) developing a treatment protocol for the patient based on results obtained in said categorizing step. In some embodiments, the immune system genes comprise genes listed in Table 4 and Table 5. In one embodiments, the pattern of gene expression of immune system genes shows upregulation of one or more genes listed in Table 5 and downregulation of one or more genes listed in Table 4, the patient is categorized as likely to undergo a relapse, and the treatment protocol developed in said developing step is an aggressive treatment protocol. In another embodiment, the pattern of gene expression of immune system genes shows upregulation of one or more genes listed in Table 4 and downregulation of one or more genes listed in Table 5, the patient is categorized as unlikely to undergo a relapse, and the treatment protocol developed in said developing step is a non-aggressive treatment protocol.

The invention also provides a system for determining a probability of relapse of a patient with a tumor. The system comprises 1) means for obtaining measurements of expression of immune system genes in tumors; 2) means for recognizing, using the measurements, patterns of gene expression, the patterns of gene expression being correlated with the probability of relapse; and 3) means for assigning a probability of relapse to the patient with said tumor. In some embodiments, the immune system genes comprise genes listed in Table 4 and Table 5.

The invention also provides a microarray chip for analyzing the likelihood of relapse of a patient with a tumor. The chip includes primers specific for amplifying RNA corresponding to one or more, or various subsets of, or all of the genes listed in Table 4 and Table 5.

### BRIEF DESCRIPTION OF THE DRAWINGS

FIGS. 1A and B. T cells derived from wild-type FVB mice will induce apoptosis in MMC in vitro but fail to reject MMC in FVBN202 mice following AIT. A) Flow cytometry analysis of MMC after 24 hrs culture with splenocytes of FVB mice following three color staining. Gated neu positive cells were analyzed for the detection of annexin V+ and PI+ apoptotic cells. Data are representative of quadruplicate experiments. B) Donor T cells were enriched from the spleen of FVB mice using nylon wool column following the rejection of MMC. FVBN202 mice (n=4) were injected with CYP followed by inoculation with MMC (4×106 cells/mouse), and tail vein injection of donor T cells. Control groups were challenged with MMC in the presence or absence of CYP treatment. Tumor growth was monitored twice weekly.

FIG. 2A-C. Gene expression profiling and gene oncology pathway analyses in tumor regressing and tumor non-regressing groups. (A) Unsupervised cluster visualization of genes differentially expressed among regressing tumors (lanes 9-12) and non regressing tumors (lanes 1-8=evasion model; lanes 13-18=tolerogenic model). MMC tumors were harvested 10 days after challenge and hybridized to 36 k oligo mouse arrays. 11256 genes with at least 3-fold ratio change and 80% presence call among all samples were projected using log 2 intensity. (B) Supervised cluster analysis (Student t test, p<0.001 and fold change>3) comparing regressing tumors (lanes 9-12) and non regressing tumors (lanes 1-8=evasion model; lanes 13-18=tolerogenic model). 2449 differentially expressed genes have been selected for further analysis. (C) Gene Ontology databank was queried to assign genes to functional categories and upregulated genes within the tumor regression group to functional categories. A=cytokine-receptor interaction; B, neuroactive ligand-receptor interaction; C=mitogen-activated protein kinase (MAPK) signaling pathway; D=regulation of actin cytoskeleton; E=cell adhesion molecules; F=natural killer mediated cytotoxicity; G=axon guidance; H=calcium signaling pathway; I=T cell receptor signaling pathway; J=insulin signaling pathway; K=Janus kinases, signal transducers and activators of transcription (JAK-STAT) signaling pathway; L=leukocyte transendothelial migration; M=Toll-like receptor signaling pathway.

FIG. 3A-C. Gene expression profiling and gene oncology pathway analyses in tolerance and evasion models. (A) Supervised cluster analysis (Student t test, p<0.001 and fold change>3) comparing evasion (Lanes 1-8) and tolerogenic group (Lanes 13-18). 1326 differentially expressed genes have been visualized including also tumor regression samples (Lanes 9-12). Gene ontology pathway analysis projecting either upregulated pathways in the evasion group (B, 854 genes, where A=focal adhesion; B=MAPK signaling; C=regulation of actin cytoskeleton; D=cell cycle; E=cytokine-receptor interaction; F=leukocyte transendothelial migration; G=axon guidance; H=small cell lung cancer; I=gap junction; J=p53 signaling pathway; K=calcium signaling pathway; L=cell communication; and M=glycan structures-biosynthesis); 1) or tolerogenic (immune suppression) tumor models (C, 475 genes, where A=cell communication; B=cell adhesion molecules, CAMs; C=insulin signaling pathway; D=cytokine-receptor interaction; E=extracellular matrix (ECM) receptor interaction; F=focal adhesion; G=tight junction; H=glycan structures biosynthesis 1; I=peroxisome proliferator-activated receptors (PPAR) signaling pathway; J=glutathione metabolism; K=glycan structures-biosynthesis 2; L=glycolysis/gluconeogenesis; and M=JAK-STAT signaling pathway).

FIG. 4A-C. Representation in tabular form of: A, chemokines and their receptors and interferon stimulating genes differentially expressed in rejection model vs control; B, cytokines and signaling molecules (interleukins and receptors, cytotoxic and pro-apoptotic molecules, Toll-like receptors and lymphocyte signaling, FC-type receptors and immunoglobulins) differentially expressed in rejection model vs control; C, genes with immunological function (chemokines, interleukins and signaling genes, and ISGs) manually selected based on supervised comparison of evasion and tolerogenic (immune suppressed) tumor models and tumor rejection model.

FIGS. 5A and B are high level flow diagrams of processes of this invention implemented on a computer.

FIG. 6. Schematic representation of the system of the invention.

FIG. 7. Results of unsupervised clustering of gene expression of 9797 genes from tumor samples of human breast cancer patients. These genes exhibit at least a 3-fold ratio in change and 80% presence (average corrected) compared to control samples.

FIG. 8. Student's T test comprising relapse vs relapse free (P<0.001) (80% corrected) of 349 genes from tumor samples of human breast cancer patients.

FIG. 9A-L. Listing, as Table 4, of 299 genes, the upregulation of which is associated with decreased occurrence of human breast cancer relapse after treatment.

FIG. 10A-B. Listing, as Table 5, of 50 genes, the upregulation of which is associated with increased occurrence of human breast cancer relapse after treatment.

FIG. 11. Significant canonical pathway analysis of immune system related pathways involved in breast cancer relapse or resistance to relapse. Solid bars=−log p value of the significance for genes upregulated in tumor lesions of patients who are relapse free vs relapsed patients, with cutoff of the significance P<0.001 (dotted line); ▪'s connected by solid lines=ratio of number of genes in relapse free vs relapsed patients. Genes which inhibit effector immune responses are underlined.

FIGS. 12A-E. Immune system pathways identified as involved in cancer relapse. A, B cell development pathway; B, antigen presentation pathway; C, graft vs host disease signaling; D, interferon signaling; and E, primary immunodeficiency signaling. The degree of gray shading of individual pathway components indicates the relative level of upregulation, with darker shading corresponding to a higher level of upregulation.

### DETAILED DESCRIPTION

The invention provides real-time identification of genetic “signatures” at tumor lesions that can be used to predict future tumor rejection and/or lack of recurrence, or failure in tumor rejection, and likely recurrence. The elucidation of differential patterns of immune system gene expression as described herein permit the classification of patients into either the category of patients who are likely to have a recurrence of the tumor, or the category of patients who are not likely to have a recurrence. 299 genes (listed in Table 4) have been identified as upregulated in breast cancer patients who do not experience recurrence after initial cancer treatments, and 50 genes (listed in Table 5) have been identified as upregulated in breast cancer patients who do experience recurrence after initial cancer treatments. An analysis which measures mRNA expression of these 349 genes thus permits recognition of patterns of gene expression (also referred to as genetic or transcription “signatures”) associated with increased or lowered risks of relapse, and can serve as a guide for health practitioners when prescribing treatment for the patient.

Thus, it was found that gene expression patterns in individuals that could successfully reject tumor recurrence showed differential expression of about 349 genes. Of these, expression of 299 genes (listed in Table 4) was increased (upregulated) in patients who did not experience relapse whereas expression of 50 genes (listed in Table 5) was downregulated in these patients, compared to normal control values. Conversely, expression of the 50 genes was upregulated in patients who did relapse and expression of the 299 genes was downregulated, compared to normal control gene expression levels. Thus, the patterns of gene expression for these two populations of patients are different or distinct from each other, and from normal control patterns. The patterns of gene expression may be referred to herein as “differential”. By “relapse” we mean that the patient, after completing customary cancer treatment (e.g. surgical tumor removal, radiation therapy, chemotherapy, etc.) and usually after being declared generally free of cancer, experiences a regrowth or reappearance of the tumor, either at the same location, or at a different location (i.e. metastatic spread of the tumor) usually within 1-5 years of completing cancer treatment. Recurrence may be due to the development of new tumor cells arising during or after treatment, or the persistence of residual tumor cells which escape the treatment that was provided.

The analysis of the invention may be carried out at any time during the life of a cancer patient, e.g. soon after diagnosis of a tumor as malignant, and prior to initial treatment, since the results provide useful information to the clinicians who develop treatment protocols. A patient who is determined to be at high risk for recurrence would generally be treated more aggressively than would a patient who is identified as at low risk for recurrence. However, the test described herein can be carried out at any point during treatment, e.g. at any time during treatment or at any time after treatment or neoadjuvant therapy as long as tumor sampling is feasible. The analysis may be carried out multiple times for a patient, e.g. in order to check whether or not the gene expression status of the patient is constant.

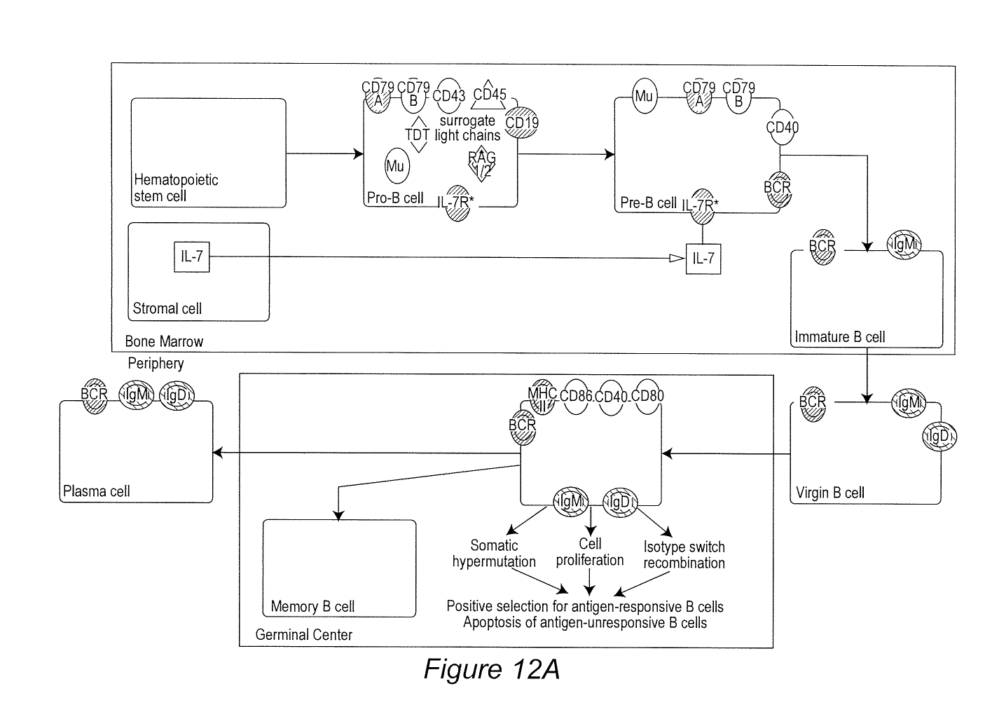

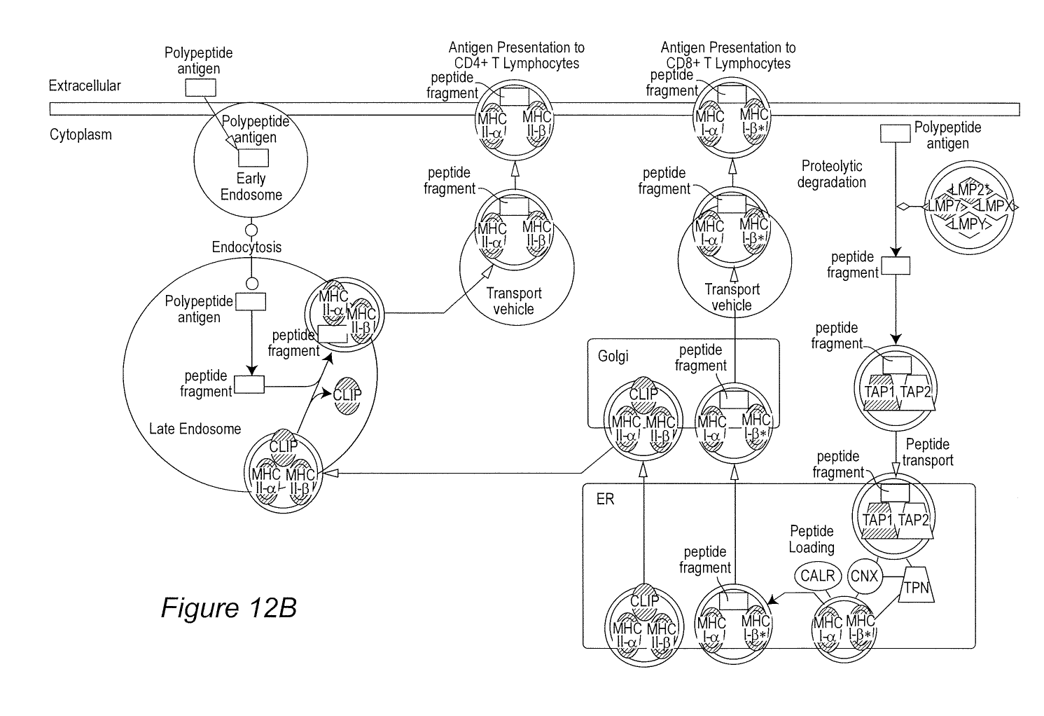

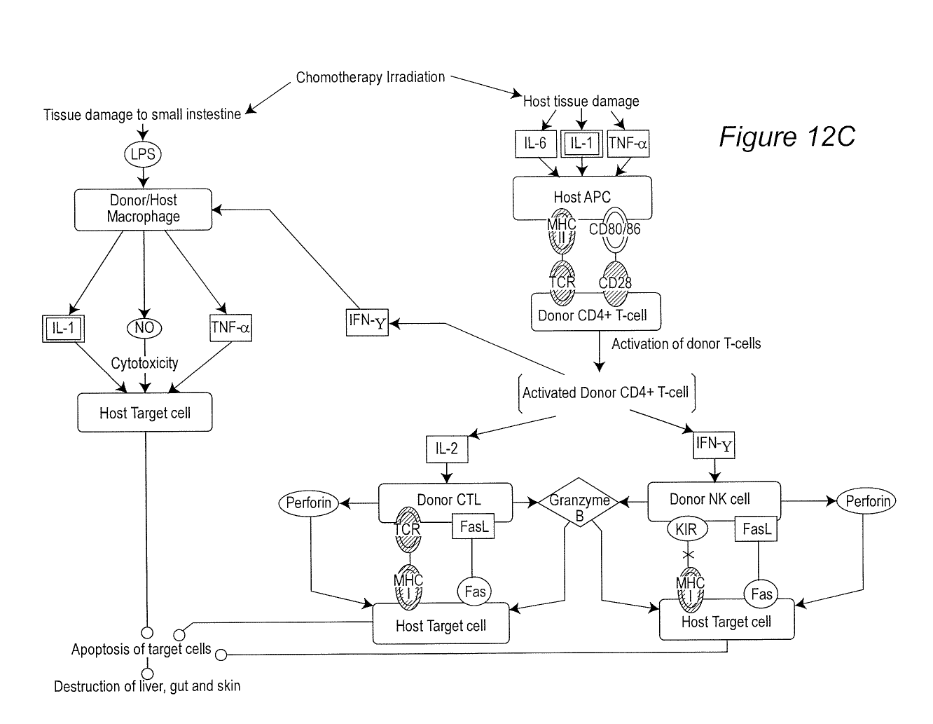

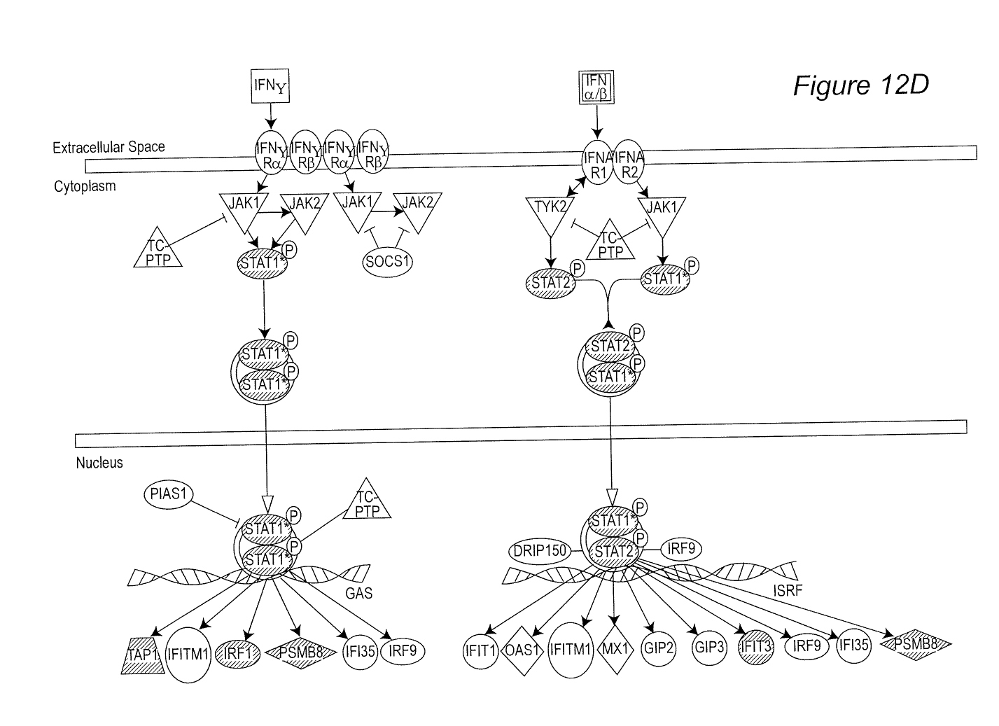

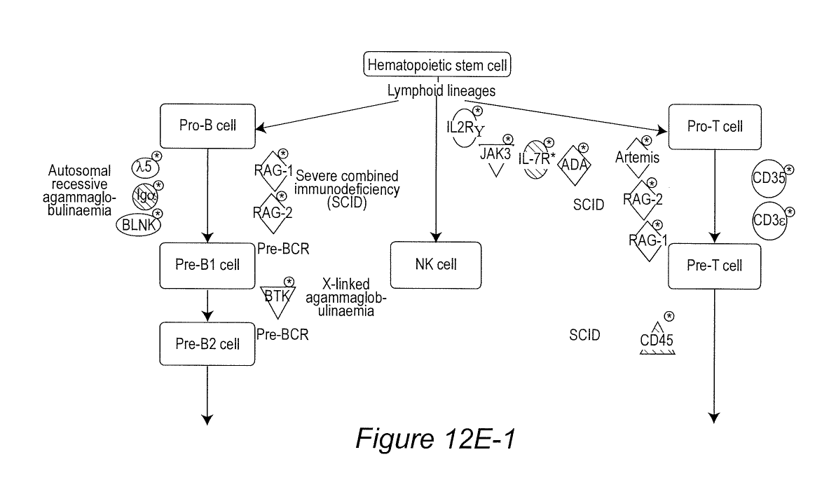

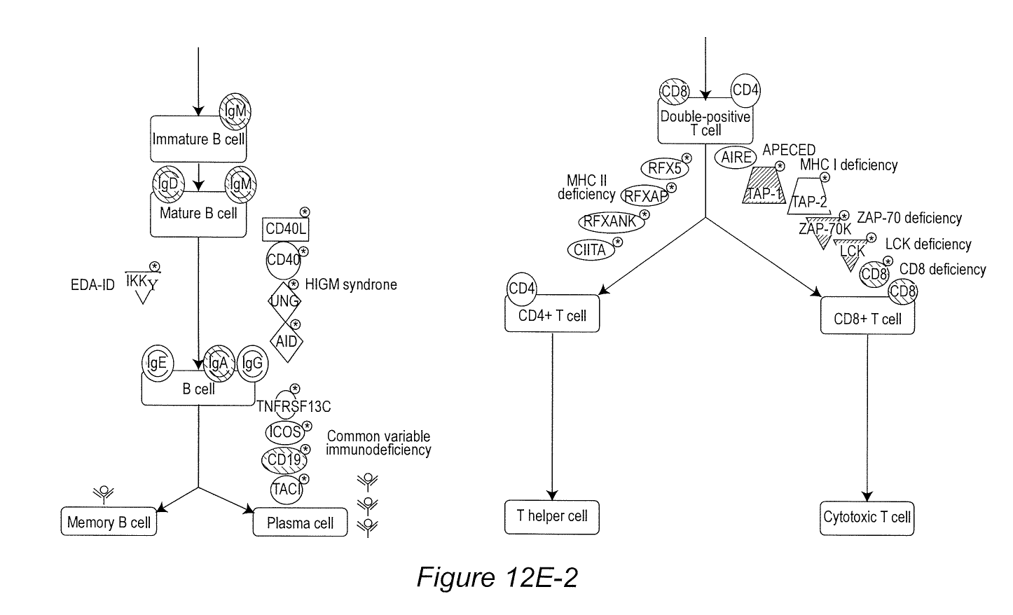

The numerical cutoffs or guidelines for assigning a patient to a low-risk vs high risk group with respect to relapse is as follows: identification of a patient as being at high risk for recurrence is indicated when the patient's gene expression profile falls in a group of 50 genes upregulated (p<0.001) in the reference relapsed group (Table 5). In other words, in a high risk patient, canonical pathway analysis would not show upregulation of the five identified immune system pathways depicted in FIGS. 12 A-E, but would show downregulation of the 299 genes listed in Table 4. Conversely, identification of a patient as being at low risk for recurrence is indicated when the individual's gene expression profile falls in a group of 299 genes upregulated (p<0.001) in the reference relapse-free group. In other words, in a low risk patient, canonical pathway analysis would show upregulation of the five identified immune system pathways, as well as downregulation of the 50 genes listed in Table 5. Generally, the level of up- or downregulation is at a level which is at least 10%, 20%, 30%, 40%, 50%, 60%, 60%, 70%, 80%, 90%, or even 100% higher or lower, respectively, than that of the reference control, and may be even 2, 3, 4, 5, 6, 7, 8, 9, or 10-fold higher or lower, respectively, or even greater, e.g. 20, 30, 40, 50, 60, 70, 80, 90, 100, or more (e.g. 150, 200, 250, 500, 750, 1000, etc.) fold higher or lower, respectively, than the level of expression of a suitable control sample(s). These gene signatures/pathways were validated by detection of the selected genes by real-time RT-PCR and Immunohistochemistry (IHC). Herein, the tendency of a person to experience relapse may be referred to as the probability, likelihood, risk chance, etc. of a recurrence or relapse.

Many of the differentially expressed genes identified herein are associated with immune regulatory functions. For example, the identified genes are generally immune system genes that are part of or are associated with an immune system pathway such asB cell development, antigen presentation, graft vs host disease signaling, interferon signaling and primary immunodeficiency signaling.

Generally, all 349 genes are analyzed in an assay, although this need not always be the case. The invention also encompasses assays in which fewer than the 349 are analyzed, or in which more than the 349 are analyzed. However, generally gene expression of at least one gene from each of the two categories (low risk, Table 4, and high risk, Table 5) is determined and the levels are compared to each other, and to the level of expression in normal control non-tumor peripheral blood mononuclear cells (PBMC). In some embodiments, at least about 10, 20, 30, 40, 50, 60, 70, 80, 90, 100, 110, 120, 130, 140, 150, 160, 170, 180, 190, 200, 210, 220, 230, 240, 250, 260, 270 280, 290, 300, 310, 320, 330, or 340 total genes are included; with at least about 10, 15, 20, 25, 30, 35, 40, 45, or all 50 genes from the group of Table 5; and at least about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or all 299 genes of the group of Table 4; or any desirable number of genes from each group.

Those of skill in the art will recognize that tumor samples (usually from solid tumors) generally contain both tumor cells and cells from the host, e.g. cells from the host immune system, blood vessels, etc. that have invaded the tumors. The “microenvironment” of a tumor, as used herein, includes such host-derived cells. As used herein, a “tumor sample” is understood to include cells from the microenvironment of the tumor and/or from the tumor itself.

Many methods for determining patterns or signatures of gene expression (e.g. differential gene expression) are known. Preferred methods include using “chip” technology, e.g. microarrays of a nucleic acid (usually DNA, although RNA is also sometimes used). DNA microarrays consist of an arrayed series of hundreds or thousands of oligonucleotide probes which hybridize to target nucleic acids (e.g. cDNA, cRNA, etc.) in a sample under high-stringency conditions. Probe-target hybridization is then detected and quantified by, e.g. fluorescence-based detection of fluorophore-labeled targets to determine relative abundance of nucleic acid sequences in the sample. In the practice of the present invention, such chip technologies may be used, and several commercially available generic chips are known which would be suitable, examples of which include but are not limited to the Affymetrix 0133+2 whole genome chip, and other chips designed for array analysis of tumor lesions using immunohistochemistry (IHC), immunofluorescence (IF), etc. Microarray chips may also be developed specifically for use in the invention. Such chips are designed to probe only selected genes of interest, such as immune system genes, or useful subsets thereof e.g. any combination of the genes described herein. Suitable controls would be included on such a specialty chip. However, those of skill in the art will understand that gene products (e.g. proteins, polypeptides, peptides) corresponding to the genes identified herein may also be detected and/or quantified, either as a primary method of determining relapse risk, or as confirmation of genetic analysis.

In one embodiment of the invention, a chip is developed specifically for use in the methods of the invention. Such a chip would include probes capable of hybridizing to one or more genes or RNA expressed from genes as described herein, i.e. genes listed in Tables 4 and 5. In addition, various useful subsets of the genes may be represented on a chip, and all such possible subsets are intended to be encompassed by the present invention, i.e. genes ranging in number from 1 to 349. In other words, those of skill in the art will recognize that, while generally an entire genetic profile or signature of a sample as described herein will be determined, this need not always be the case. For example, given the information provided herein, it is also possible to select certain genes or small groups of genes and compare their expression on a less comprehensive basis. All such variations and permutations of the technology disclosed herein are intended to be encompassed by this invention.

For the practice of the invention, the experimental or unknown samples for which a genetic signature is obtained are generally tumor samples such as biopsy samples, or samples of a tumor that has been surgically removed from a patient. Procedures for obtaining such samples are generally carried out by skilled medical personnel such as physicians, surgeons, etc. Likewise, the treatment and handling of tumor samples in order to extract nucleic acids such as RNA for analysis from the samples may vary somewhat from circumstance to circumstance, but such methods are generally known, e.g. homogenizing samples in the presence of various agents such as nuclease inhibitors in order to promote or preserve the stability of the mRNA, apportioning samples, purifying fractions, adding reagents such as enzymes, labeling agents, etc., which is generally carried out manually or in a partially automated manner by those skilled in laboratory procedures. Any and all such suitable methods may be used to prepare the sample for analysis.

Once the gene expression pattern of the patient is determined, it is possible to tailor the patient's treatment based on the results of the assessment, i.e. the treatment protocol of a patient may be adjusted to account for the tendency, or lack thereof, toward relapse (tumor recurrence, regrowth, or redevelopment).

For example, if the analysis suggests that the tumor is not likely to recur, non-aggressive treatment alternatives might include conservative surgery, lower frequency and duration of radiation and/or lower frequency of chemotherapy with a preference of low toxicity drugs. Alternatively, if the analysis suggests that the tumor is likely to recur, treatment with one or more of aggressive surgery, radiation and/or chemotherapy may be recommended.

Immune responses to various types of cancers can be determined by the practice of the methods of the invention. Generally, such cancers will be of the type that form solid tumors, i.e. carcinomas. Examples of such cancers include but are not limited to breast cancer, ductal carcinoma in situ (DCIS), prostate cancer, stomach cancer, colon cancer, lung cancer, melanoma, and head and neck cancer. The tumors that are analyzed may be primary or secondary (metastasized) or recurring tumors, and the methods may be used to monitor the effects of treatments and patient progress.

Patients who may benefit from the analyses described herein are generally mammals, and may be humans, although that need not be the case. Veterinary applications of this technology are also contemplated.

Those of skill in the art will recognize that comparisons and analyses of gene expression patterns such as those described herein are generally automated to the extent possible, and are controlled by computer software analytical programs. The invention also provides computer implemented methods of determining, comparing and analyzing gene expression patterns, in order to assess the likelihood of tumor relapse in a patient. The analytical programs of the invention may be interfaced with, for example, programs that are part of an automated nucleic acid detection system so that data from the automated nucleic acid detection system is fed directly to the analytical programs of the invention. For example, final identification of the genes that are expressed and measurement of the amount of gene expression products that are present in a sample is usually determined in an automated manner. Those of skill in the art will recognize that automated systems exist that are designed, once supplied with appropriate starting material (e.g. suitable nucleic acid sample to analyze, labeling reagents, etc.). Such programs are typically computer implemented and are able to output, for example, the identity of the genes associated with the nucleic acids in the sample and the quantity of the expressed genes, e.g. the degree of upregulation or downregulation. The interface between the analytical programs of such a system and those of the present invention may be direct or indirect, i.e. the programs of the invention may be merely linked to accept information from such a program, or one program with both capabilities may be developed. The analytical programs of the invention may contain (and in fact may be used to develop or update) a database of gene expression patterns from tumors (either from a specific individual, or from a plurality of individuals) and usually also have the capability to compare the results obtained with an experimental or unknown sample to the results of reference or control values in the database, or to any other value in the database. The computer programs are generally capable of statistically analyzing the data, including determining the significance of deviations from normal or control values, or between samples, or between sets of genes from a sample e.g. to determine differential expression of genes between the genes listed in Table 4 and those listed in Table 5.

The output of the programs of the invention may include, for example, absolute or relative levels of gene expression, e.g. identification of the expression of one or more genes of interest, identification of the absence of expression of one or more genes of interest, the levels of expression of one or more genes of interest (e.g. percentages, fold increases or decreases, etc.), and the like. In addition, the computer implemented analytical methods of the invention may interface directly or indirectly with cancer treatment protocol programs, i.e. two programs may be linked to merely accept data or output from another program, or one program encompassing both capabilities may be developed. In addition, all three programs (analysis of gene patterns, prognosis of patient response to tumor, and suggested treatment protocols) may be linked or integrated into one computer-implemented program. In this case, output from the program may be one or more suggested courses of treatment (treatment protocols) for the patient from whom the tumor sample was obtained.

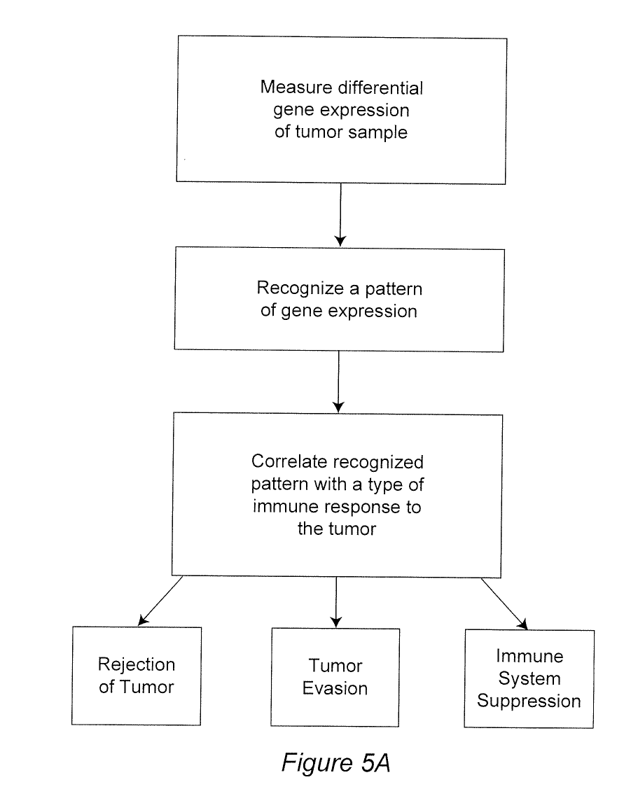

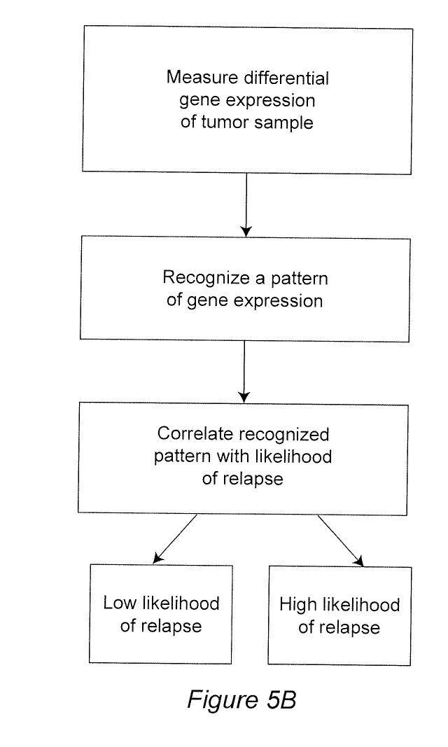

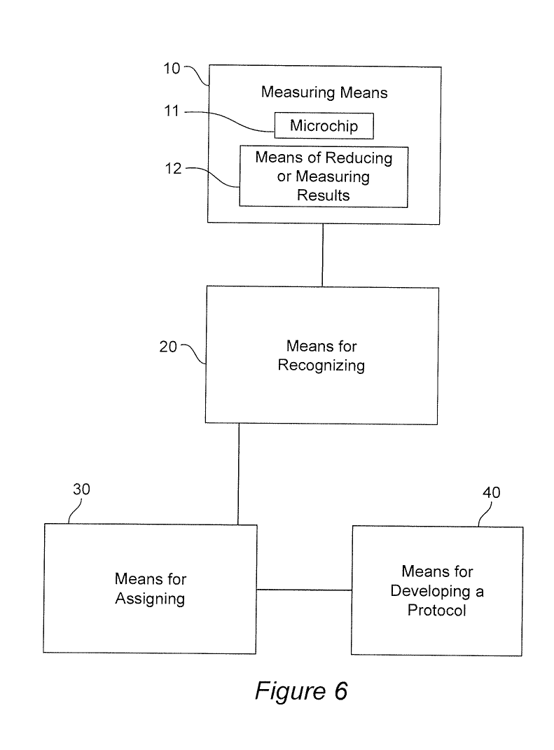

The invention also provides a system for characterizing an immune response of a patient with a tumor. A flow chart of the basic steps of one embodiment of the method is provided in FIG. 5A and another is illustrated in FIG. 5B. The system is illustrated schematically in FIG. 6. The system includes measuring means 10 for obtaining measurements of differential expression of immune system genes in tumors. Those of skill in the art will recognize that such a system may include a microchip or “genechip” 11 for hybridizing nucleic acids from the sample of interest (in this case, a tumor sample), and that results from microchip hybridization experiments may be converted into a detectable signal, such as a fluorescent, luminescent, or other type of signal. The means for obtaining measurements may also comprise various detectors or other means of reading or measuring results 12 obtained with the microchip. Generally, the results will be expressed in the form of numeric values corresponding to levels of expression of the genes that were tested, e.g. lists of the amounts or relative amounts of detected transcription products associated with the genes of interest (e.g. immune system genes as described herein). Statistical significance data may also be provided.

The system of the invention also includes means for recognizing 20, using the measurements, patterns of differential gene expression. The means for recognizing 20 will generally be a computer processor or a network of computers comprising a computer implemented program (e.g. software with instructions, enclosed in a computer readable medium such as a diskette, hard disk, CD ROM, DVD, thumb drive, firmware, etc.) capable of receiving (inputting) the measurements from measuring means 10, and capable of statistically analyzing the measurements to identify or recognize one of three prototypic patterns, each of which correlates with one of the following three types or categories of immune responses: i) an immune response that is rejecting said tumor; ii) an immune response that is not rejecting said tumor due to the action of immune suppressing factors; and iii) an immune system response that is not rejecting said tumor due to changes in said tumor. The means for recognizing 20 can also output (i.e. comprises a means to output) the recognized pattern for display, for further processing and analysis, etc.

The system of the invention also includes means for assigning 30, capable of receiving the recognized pattern from recognizing means 20, and, based on the pattern, assigning a characteristic of interest (e.g. proclivity toward tumor recurrence, or lack thereof) to the particular patient from whom the tumor sample was obtained. Assigning means 30 may also be a computer (the same or different from those described previously) comprising a computer implemented program (e.g. software, etc. as described previously) capable of receiving (inputting, i.e. containing a means to input) the pattern recognized by recognizing means 20. (In fact, recognizing means 20 and assigning means 30 may be integrated into a single computerized system.) This assignation can be outputted via an output means to a user, and used to establish a suitable treatment protocol. In fact, the system may optionally further include a means for developing and outputting a recommended treatment protocol 40 (which may or may not be integrated into a single computerized system with recognizing means 20 and assigning means 30). In all cases, output from each system means may be electronic (e.g. input or downloaded to another instrument, or to a computer screen or display). Alternatively, or in addition, hard copies of the output may be generated, e.g. by a printer that is linked to the system. The output may be in the form of, for example, a list, chart, diagram, photograph or photograph-like digitalized reproduction of results, and the like.

Those of skill in the art will recognize that instructions for causing a computer to carry out the computer-implemented analysis programs for one or more of measuring differential gene expression, recognizing a gene expression pattern as described herein and assigning or associating a pattern to/with a particular type of outcome, (and optionally for developing a treatment protocol) may be integrated into a single computer program or firmware.

The results obtained from the system of the invention are used or interpreted by a health care professional (e.g. a physician, or other skilled professional) to plan, recommend, adjust, modify or otherwise develop a treatment program that is tailored to the needs of the patient with the tumor. The treatment that is recommended is based on or takes into account the results of the analysis. Such a treatment will be more likely to provide benefit to the patient by working with or taking into account the patient's immune response or the status of the patient's immune system, rather than possibly aggravating the patient's immune response to the tumor, or rather than treating the tumor according to a protocol that does not take individual patient differences into account. For example, for patients who are likely to relapse, an aggressive treatment stance may be taken, including aggressive surgery and prolonged chemotherapy, radiation therapy, etc. In contrast, for patients who are unlikely to relapse, may receive more conservative, less aggressive treatments.

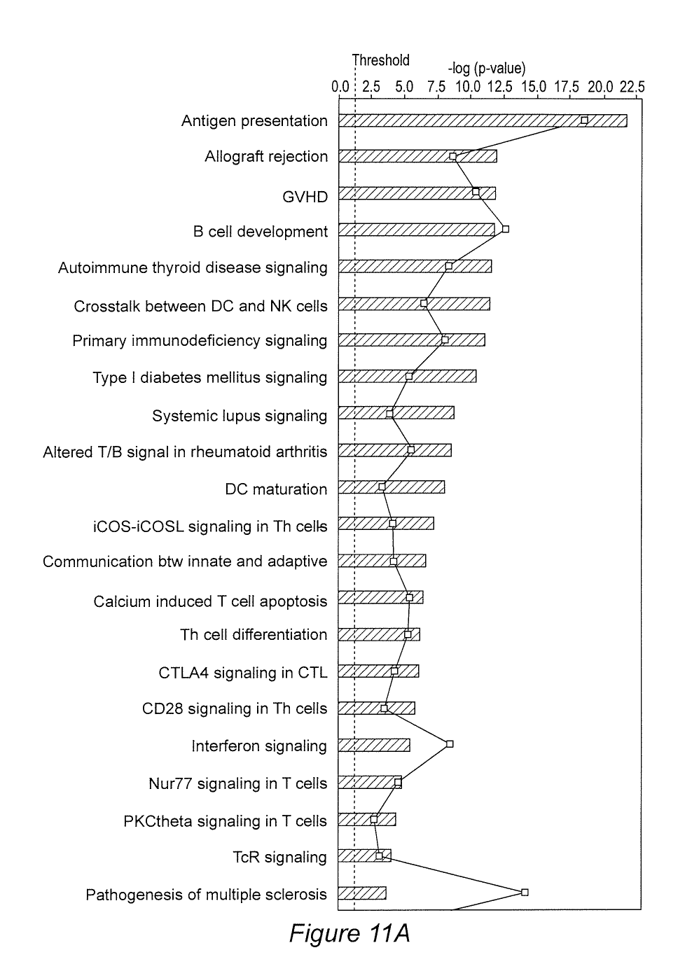

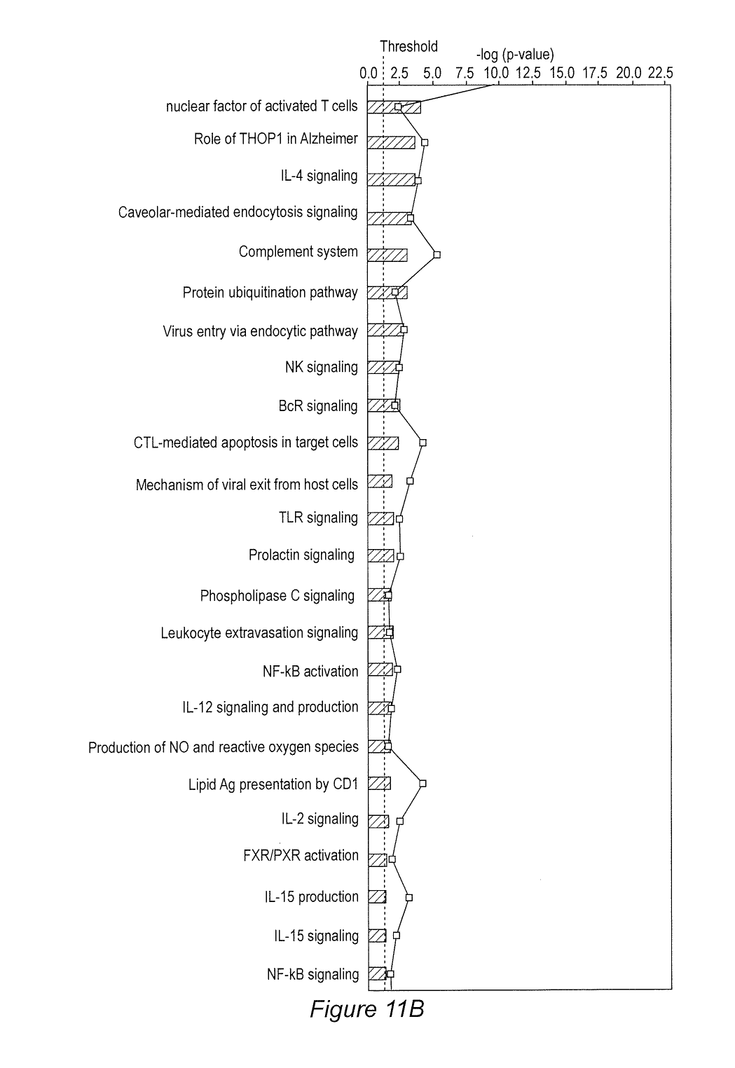

Like breast cancer patients, distinct signatures of immune function genes associated with recurrence or recurrence-free survival can be identified in mouse model of breast carcinoma, though with different pattern of gene signatures. In some embodiments, failure to reject tumor cells may be due to changes in the phenotype of the tumor itself, which allow it to evade the patient's immune system response, which is essentially normal and unimpaired. In this case, the patient's immune system initially mounts an appropriate response and, as the tumor changes, continues to attempt to deal with the tumor e.g. by switching from a predominantly Th-1 response (increased ISGs and granzyme) to a Th-2/humoral response. However, Th-2 and humoral responses are less successful in eradicating cancer cells, and rejection of tumor cells that arise after initial treatment does not occur, so a recurrence of another iteration of the tumor is likely. On the other hand, failure to reject may be due to direct suppression of the patient's immune system. In this scenario, immune suppressing factors (usually secreted by tumor cells) act on the immune system cells of the patient, causing it to shut down. These genes/pathways are shown in FIG. 11 and include primary immunodeficiency signaling, calcium induced T cell apoptosis, CTLA4 signaling in CTL, production of NO and reactive oxygen species. Without being bound by theory, it is possible that upregulation of these genes even in relapse free patients may explain why these patients failed to reject their tumors in the first place, and that when the tumor is removed by surgery, these immune suppressors that are induced by tumor-derived factors will disappear and other immune effector genes will then be able to destroy residual tumors. In other words, in this scenario, primary rejection does not occur, and technically recurrence also does not occur since, from the beginning, the patient's immune effectors are suppressed, though are still present. At one time, it was thought that such a lack of rejection was due to “tolerance” of the patient's immune system for the tumor. However, it has now been discovered that tolerance to tumors never exists in cancer patients; a patient's immune system does not simply ignore or not respond to the presence of tumor cells. Instead, failure to reject tumors is due to the induction of immune regulatory mechanisms that suppress anti-tumor immune responses. These findings change the existing paradigm for understanding tumor formation in cancer patients.

In contrast, different patterns of gene expression were observed in both categories of non-rejected tumors. In the case in which tumor evasion has occurred or is occurring, the Chemokines such as those listed in Table 3 are differentially expressed compared to individuals who reject the tumor or individuals whose immune system is suppressed. It is noted that some molecules such as Cxcl9 are increased in the rejection and recurrence models compared with the “tolerogenic” (suppression) model; this indicates that tumor recurrence has occurred under immune pressure such that the anti-tumor immune response rejected HER-2/neu positive tumors and at the same time induced loss of HER-2/neu and resulted in the recurrence of HER-2/neu negative tumor variants. In addition, when tumor evasion occurs, various Interleukins and Signaling genes such as those listed above the top-most solid line in Table 3 are differentially expressed. Further, when tumor evasion occurs, ISGs listed above the second (bottom) heavy line in Table 3 were also differentially expressed. Basically, when tumor evasion occurs, the host immune response contains some hallmarks of a Th-1 response, together will elements of a Th-2 and/or humoral response.

In the case of immune suppression, the pattern of gene expression is generally characterized by reduced Cxcl9 expression. Expression fo some interleukins and signaling molecules such as Vpreb3 and Pias2 are also reduced, but those below the first (top-most) heavy line of Table 3 show increased expression compared to immune suppressed individuals, and, with some exceptions, in comparison to individuals who reject the tumor. Lastly, ISGs listed below the lower heavy line in Table 3 are generally upregulated in immune suppression individuals, compared to tumor evasion individuals, and with some exceptions, in comparison to individuals who rejected the tumor as well. Those of skill in the art will recognize that in some cases, the pattern of gene expression will be increased above a reference level whereas in other cases the pattern may show a decrease to below a reference level. Both of these deviations from the reference are valuable and can form a part of the overall genetic signature of the model. Similarly, some increases or decreases may overlap across models, e.g. may be increased in two out of the three and decreased in only one. Nevertheless, such markers are valuable because the genetic signature is based on the assessment of results obtained with many genes, and it is the overall patterns that are characteristic of a condition of interest.

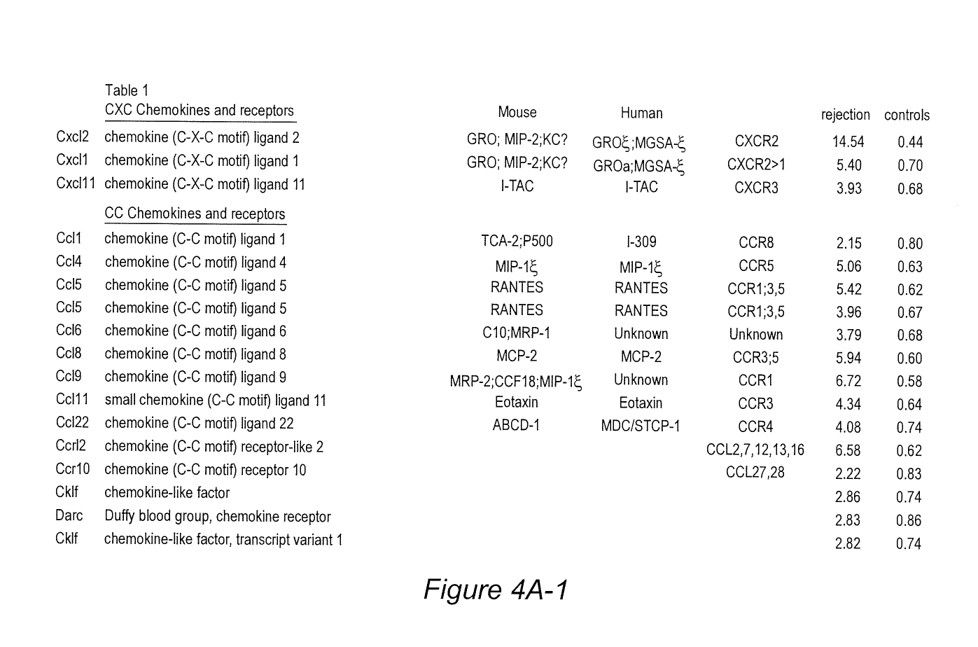

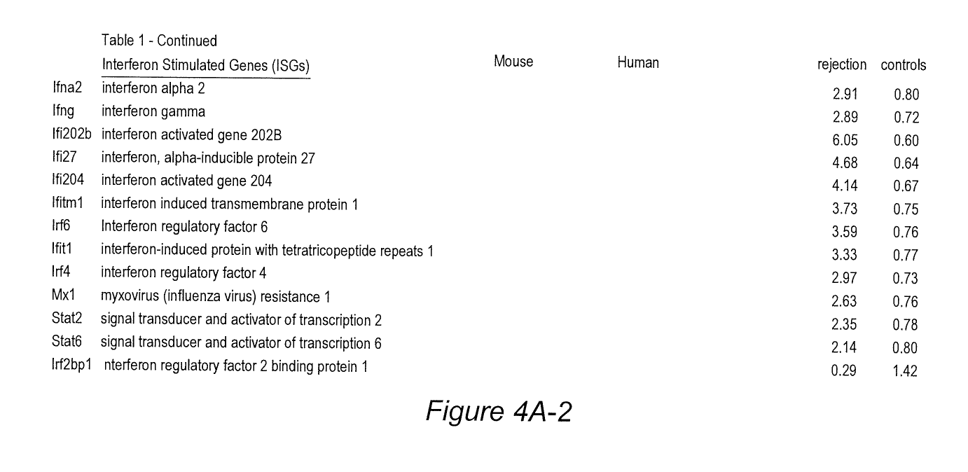

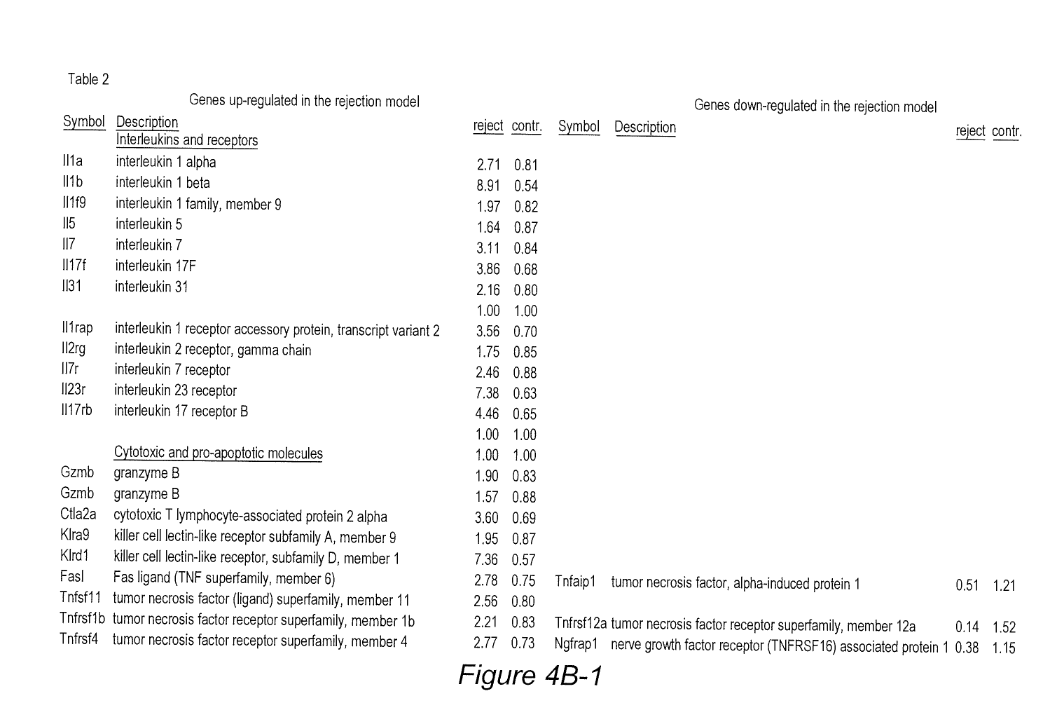

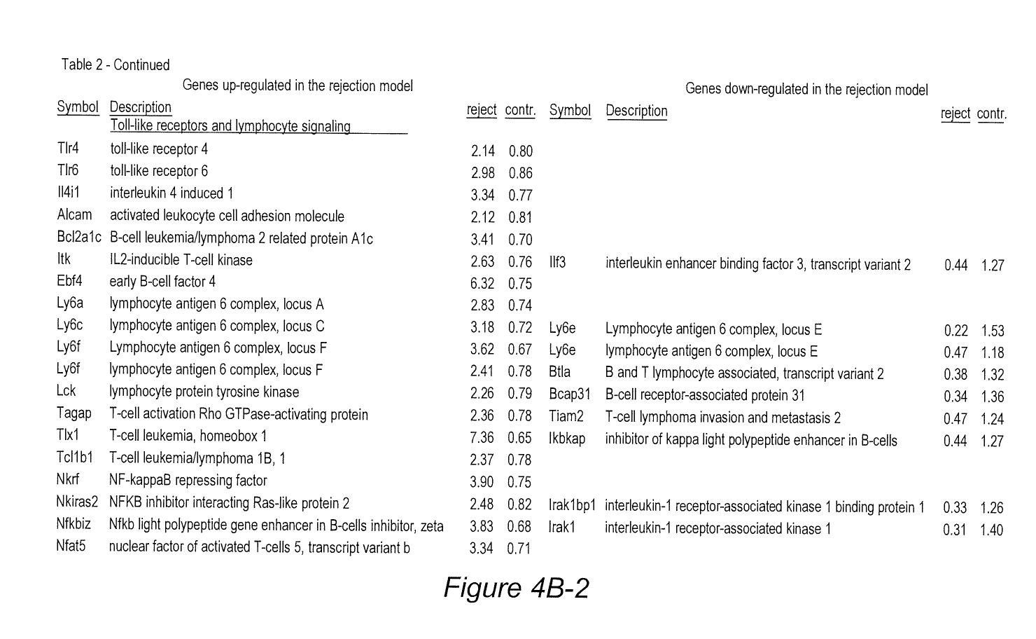

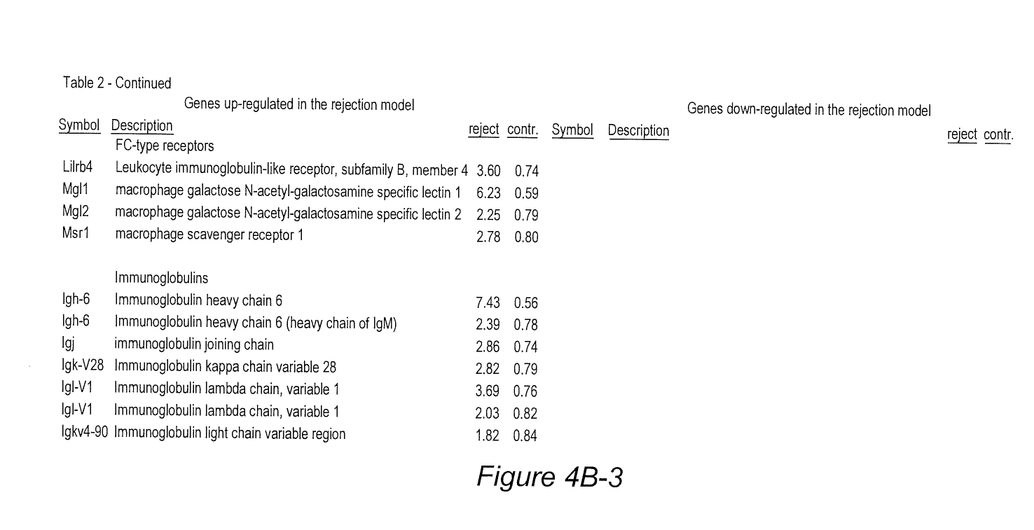

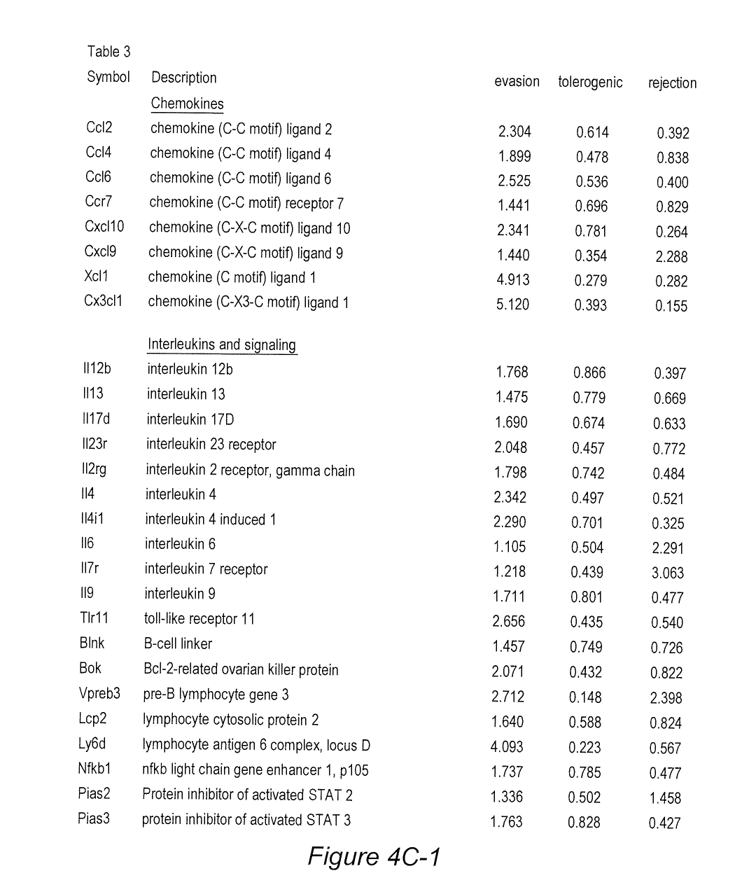

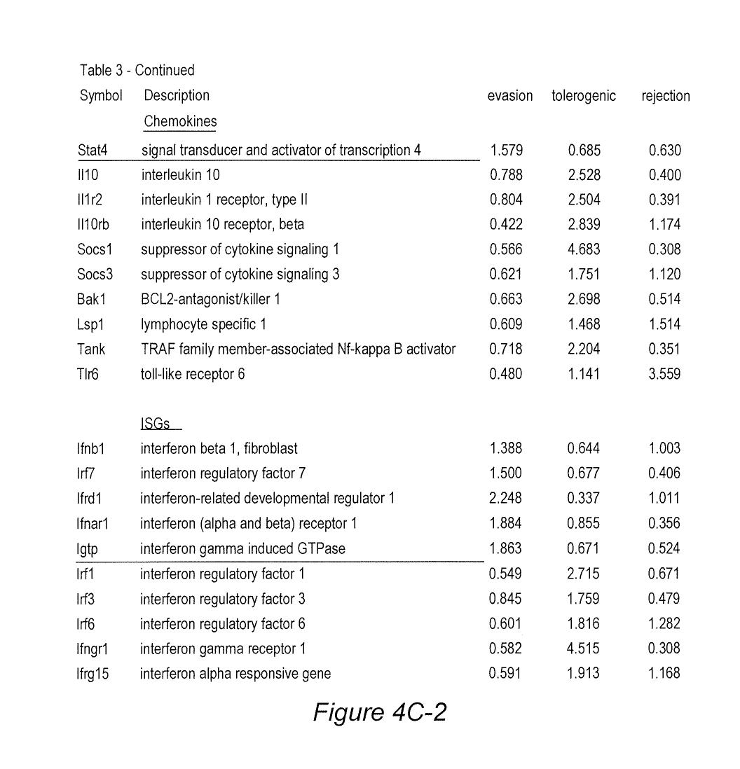

These differentially expressed genes can be organized into three categories: 1) chemokines; 2) interferon-stimulated genes (ISGs); and 3) cytokines and signaling molecules. Table I (presented as FIG. 4A) lists exemplary chemokines (and receptors) and exemplary ISGs and Table 2 (presented as FIG. 4B) lists exemplary cytokines and signaling molecules which may be differentially expressed in a tumor microenvironment of a patient that is mounting a robust, appropriate immune response to the tumor. Table 3 (presented as FIG. 4C) lists other selected genes of interest with immunological function.

### I. CHEMOKINES

Exemplary chemokines and chemokine related molecules such as receptors include but are not limited to:

CXC chemokines and receptors such As: chemokine (C-X-C motif) ligand 2 (Cxcl 2), chemokine (C-X-C motif) ligand 1 (Cxcl 1) and chemokine (C-X-C motif) ligand 11 (Cxcl 11).

CC chemokines and receptors such as: chemokine (C-C motif) ligand 1 (Cell); chemokine (C-C motif) ligand 4 (Ccl4); chemokine (C-C motif) ligand 5 (Ccl5); chemokine (C-C motif) ligand 6 (Ccl6); chemokine (C-C motif) ligand 8 (Ccl8); chemokine (C-C motif) ligand 9 (Ccl9); chemokine (C-C motif) ligand 11 (Ccl11); chemokine (C-C motif) ligand 22 (Ccl22); chemokine (C-C motif) receptor-like 2 (Ccrl 2); chemokine (C-C motif) receptor 10 (Ccrl 10); chemokine-like factor (Cklf); Duffy blood group, chemokine receptor (Dare); and chemokine-like factor, transcript variant 1 (Cklf).

Chemokines such as: chemokine (C-C motif) ligand 2 (Ccl2); chemokine (C-C motif) ligand 4 (Ccl4); chemokine (C-C motif) ligand 6 (Ccl6); chemokine (C-C motif) receptor 7 (Ccr7); chemokine (C-X-C motif) ligand 10 (Cxc10); chemokine (C-X-C motif) ligand 9 (Cxc9); chemokine (Cmotif) ligand 1 (Xcl1); and chemokine (C-X3-C motif) ligand 1 (Cx3cl).

### II. INTERFERON-STIMULATED GENES (ISGs)

Exemplary Interferon stimulated genes (ISGs) include but are not limited to: interferon alpha 2 (Ifna2); interferon gamma (Ifng); interferon activated gene 202B (Ifn202b); interferon, alpha-inducible protein 27 (Ifn27); interferon activated gene 204 (Ifn204); interferon induced transmembrane protein 1 (Ifntm1); interferon regulatory factor 6 (Inf6); interferon-induced protein with tetratricopeptide repeats 1 (Ifnt1); interferon regulatory factor 4 (Ifn4); myxovirus (influenza virus) resistance 1 (Mx 1); signal transducer and activator of transcription 2 (Stat 2); signal transducer and activator of transcription 6 (Stat 6); and interferon regulatory factor 2 binding protein 1 (Ifn2 bp1).

Exemplary ISG genes also include: interferon beta 1, fibroblast (Ifnb1); interferon regulatory factor 7 (Irf7); interferon-related developmental regulator 1 (Ifrd1); interferon (alpha and beta) receptor 1 (Ifnar1); interferon gamma induced GTPase (Igtp); interferon regulatory factor 1 (Irf1); interferon regulatory factor 3 (Irf3); interferon regulatory factor 6 (Irf6); interferon gamma receptor 1 (Irfgr1); and interferon alpah responsive gene (Ifrgl5).

### CYTOKINES AND SIGNALING MOLECULES

Exemplary cytokines and signaling molecules include but are not limited to:

Various interleukins and receptors such as: interleukin 1 alpha (Ila); interleukin 1 beta (Il 1b); interleukin 1 family, member 9 (Il1 f9); interleukin 5 (Il5); interleukin 7 (Il7); interleukin 17F (IL17f); interleukin 31 (Il31); interleukin 1 receptor accessary protien; transcript variant 2 (Il1rap); interleukin 2 receptor, gamma chain (Il2rg); interleukin 7 receptor (Il7r); interleukin 23 receptor (Il23r); and interleukin 17 receptor (Il17rb).

Various cytotoxic and pro-apoptotic molecules such as: granzyme B (Gzmb); cytotoxic T lymphocyte-associated protein 2 alpha (Ctla2a); killer cell lectin-like receptor subfamily A, member 9 (Klra9); killer cell lectin-like receptor subfamily D, member 1 (Klrd1); Fas ligand (TNF superfamily, member 6) (Fas1); tumor necrosis factor (ligand) superfamily, member 11 (Tnfsfl 1); tumor necrosis factor receptor superfamily, member 1b (Tnfsf1b); and tumor necrosis factor receptor superfamily, member 4 (Tnfsf4).

Various Toll-like receptors and lymphocyte signaling molecules such as: toll-like receptor 4 (Tlr4); toll-like receptor 6 (Tlr6); interleukin 4 induced 1 (IL4i1); activated leukocyte cell adhesion molecule (Alcam); B-cell leukemia/lymphoma 2 related protein A1c (Bcl2a1c); 1L-2-inducible T-cell kinase (Itk); early B-cell factor 4 (Ebf4); lymphocyte antigen 6 complex, locus A (Ly6a); lymphocyte antigen 6 complex, locus C (Ly6c); lymphocyte antigen 6 complex, locus F (Ly6f); lymphocyte protein tyrosine kinase (Lck); T-cell activation Rho GTPase-activating protein (Tagap); T-cell leukemia, homeobox 1 (Tls1); T-cell leukiemia/lymphoma 1B, 1 (Tcl1b1); NF-kappaB repressing factor (Nkrf); NFKB inhibitor interacting Ras-like protein 2 (Nkiras2); Nfkb light polypeptide gene enhancer in B-cells inhibitor, zeta (Nlfkbiz); and nuclear factor of activated T-cells 5, transcript variant b (Nfat5);

Various FC-type receptors such as: Leucocyte immunoglobulin-like receptor, subfamily B, member 4 (Ilrb4); macrophage galactose N-acetyl-galactosamine specific lectin 1 (Mgl1); macrophage galactose N-acetyl-galactosamine specific lectin 2 (Mgl2); and macrophage scavenger receptor 1 (Msr1).

Various immunoglobulin genes such as: immunoglobulin heavy chain 6 (Igh-6); immunoglobulin heavy chain 6 (heavy chain of IgM) (Igh-6); immunoglobulin joining chain (Igj); immunoglobulin heavy chain 6 (Ign-6); immunoglobulin kappa chain variable 28 (Igk-V28); immunoglobulin lambda chain, variable 1 (Igl-V1); and immunoglobulin light chain variable region (Igkv4-90).

Various interleukin and signaling genes such as: interleukin 12b (Il12b); interleukin 13 (Il13); interleukin 17D (Il17d); interleukin 23 receptor (Il23r); interleukin 2 receptor, gamma chain (Il2rg); interleukin 4 (Il4); interleukin 4 induced 1 (IL4i1); interleukin 6 (Il6); interleukin 7 receptor (Il7r); interleukin 9 (Il9); toll-like receptor 11 (Tlr11); B-ce. Linker (Blnk); Bcl-2-related ovarian killer protein (Bok); pre-B lymphocyte gene 3 (Vpreb3); lymphocyte cytosolic protein 2 (Lcp2); lymphocyte antigen 6 complex, locus D (Ly6d); mfkb light chain gene enhancer 1, p105 (Nfkb1); protein inhibitor of activated STAT 2 (Pias2); protein inhibitor of activated STAT 3 (Pias3); signal transducer and activator of transcription 4 (Stat 4); interleukin 10 (Il10); interleukin 1 receptor, type II (Il1r2); interleukin 10 receptor, beta (IL10b); suppressor of cytokine signaling 1 (Socs1); suppressor of cytokine signaling 3 (Socs3); BCL-2-antagonist/killer 1 (Bak1); lymphocyte specific 1 (Lsp1); TRAF family member-associated Nf-kappa B activator (Tank); and toll-like receptor 6 (Tlr6).

The invention is further illustrated in the ensuing examples, which are provide to illustrate the invention but which should not be interpreted as limiting the invention in any way.

### EXAMPLES

### Example 1

### Signatures associated with rejection or recurrence in HER-2/neu positive mammary tumors

Summary: We have previously shown T cell-mediated rejection of the neu-overexpressing mammary carcinoma cells (MMC) in wild-type FVB mice. However, following rejection of primary tumors, a fraction of animals experience a recurrence of a neu antigen-negative variant (ANV) of MMC (tumor evasion model), after a long latency period. In the present study, we determined that T cells derived from wild-type FVB mice can specifically recognize MMC by secreting IFN-γ and can induce apoptosis of MMC in vitro. Neu-transgenic (FVBN202) mice develop spontaneous tumors that they cannot reject (tumor tolerance model). To dissect the mechanisms associated with rejection or tolerance of MCC tumors, we compared transcriptional patterns within the tumor microenvironment of MMC undergoing rejection with those that resisted rejection either because of tumor evasion/antigen-loss recurrence (ANV tumors) or because of intrinsic tolerance mechanisms displayed by the transgenic mice. Gene profiling confirmed that immune rejection is primarily mediated through activation of interferon stimulated genes (ISGs) and T cell effector mechanisms. The tumor evasion model demonstrated combined activation of Th1 and Th2 with a deviation towards Th-2 and humoral immune responses that failed to achieve rejection likely because of lack of target antigen. Interestingly, the tumor tolerance model instead displayed immune suppression pathways through activation of regulatory mechanisms that included in particular the over-expression of IL-10, IL-10 receptor and suppressor of cytokine signaling (SOCS)-1 and SOCS-3. This data provides a road-map for the identification of novel biomarkers of immune responsiveness in clinical trials.

In order to carry out this work, we adopted an experimental model that could address the paradoxical relationship between adaptive immune responses against cancer antigens and rejection or persistence of antigen-bearing cancers with the intent of comparing functional signatures between the experimental model and previous human observation that could shed mechanistic information on this relationship and potentially provide novel predictive or prognostic biomarkers to be tested in the clinical settings. In this study, we compared transcriptional patterns of mammary tumors undergoing rejection to that of related tumors that evaded immune recognition through antigen loss (evasion model) or resided in tolerized transgenic mice (tolerogenic model). For this purpose, we used FVB mice that reject neu-overexpressing mammary carcinomas (MMC) because of the presence of a potent neu-specific T cell response. Although MMC are consistently rejected after a few weeks, occasionally MMC recur and in such instances they resist further immune pressure by invariably loosing HER-2/neu expression (tumor evasion model) (21, 22). Moreover, FVBN202 mice that constitutively express high levels of HER-2/neu fail to reject MMC because they cannot mount effective anti-tumor T cell responses (tolerogenic model). Thus, we compared the tumor microenvironment at salient moments of immune response/evasion/tolerance to gain, in this previously well-characterized model (21, 22), insights about the immune mechanisms leading to tumor rejection and their failure in conditions of tumor evasion or systemic tolerance. Interestingly, the tolerance model, which was expected to show tolerance, displayed immune suppression pathways through activation of regulatory mechanisms that included in particular the over-expression of IL-10, IL-10 receptor and suppressor of cytokine signaling (SOCS)-1 and SOCS-3.

### Materials and Methods

### Mice

Wild-type FVB (Jackson Laboratories) and FVBN202 female mice (Charles River Laboratories) were used throughout these studies. FVBN202 is the rat neu transgenic mouse model in which 100% of females develop spontaneous mammary tumors by 6-10 mo of age, with many features similar to human breast cancer. These mice express an unactivated rat neu transgene under the regulation of the MMTV promoter (23). Because of the overexpression of rat neu protein, FVBN202 mice are expected to tolerate the neu antigen as self protein, and in cases where there might be a weak neu-specific immune response prior to the appearance of spontaneous mammary tumors are still well tolerated (24, 25). On the other hand, rat neu protein is seen as nonself antigen by the immune system of wild-type FVB mice, resulting in aggressive rejection of primary MMC (21, 26). The studies have been reviewed and approved by the Institutional Animal Care and Use Committee (IACUC) at Virginia Commonwealth University.

Tumor cell lines.

The MMC cell line was established from a spontaneous tumor harvested from FVBN202 mice as previously described (11, 15). Tumors were sliced into pieces and treated with 0.25% trypsin at 4° C. for 12-16 h. Cells were then incubated at 37° C. for 30 min, washed, and cultured in RPMI1640 supplemented with 10% Fetal Bovine Serum (FBS) (21, 22). The cells were analyzed for the expression of rat neu protein before use. Expression of rat neu protein was also analyzed prior to each experiment and antigen negative variants (ANV) were reported accordingly (see results).

### In Vivo Tumor Challenge.

Female FVB or FVBN202 mice were inoculated s.c. with MMC (4-5×10⁶ cells/mouse). Animals were inspected twice every week for the development of tumors. Masses were measured with calipers along the two perpendicular diameters. Tumor volume was calculated by: V(volume)=L(length)×W(width)²÷2. Mice were sacrificed before a tumor mass exceeded 2000 mm³ IFN-γ ELISA

Secretion of MMC-specific IFN-γ by lymphocytes was detected by co-culture of lymphocytes (4×10⁶ cells) with irradiated MMC or ANV (15,000 rads) at 10:1 E:T ratios in complete medium (RPMI1640 supplemented with 10% FBS, 100 U/ml penicillin, 100 μg/ml streptomycin) for 24 hrs. Supernatants were then collected and subjected to IFN-γ ELISA assay using a Mouse IFN-γ ELISA Set (BD Pharmingen, San Diego, Calif.) according to the manufacturer's protocol. Results were reported as the mean values of duplicate ELISA wells.

### Flow Cytometry.

A three color staining flow cytometry analysis of the mammary tumor cells (10⁶ cells/tube) was carried out using mouse anti-neu (Ab-4) Ab (Calbiochem, San Diego, Calif.), control Ig, FITC-conjugated anti-mouse Ig (Biolegend, San Diego, Calif.), PE-conjugated annexin V and propidium iodide (PI) (BD Pharmingen, San Diego, Calif.) at the concentrations recommended by the manufacturer. Cells were finally added with annexin V buffer and analyzed at 50,000 counts with the Beckman Coulter EPICS XL within 30 min.

Microarray performance and statistical analysis:

Total RNA from tumors was extracted after homogenization using Trizol reagent according to the manufacturer's instructions. The quality of secondarily amplified RNA was tested with the Agilent Bioanalyzer 2000 (Agilent Technologies, Palo Alto, Calif.) and amplified into anti-sense RNA (aRNA) as previously described (27, 28). Confidence about array quality was determined as previously described (29). Mouse reference RNA was prepared by homogenization of the following mouse tissues (lung, heart, muscle, kidneys and spleen) and RNA was pooled from 4 mice. Pooled reference and test aRNA were isolated and amplified in identical conditions during the same amplification/hybridization procedure to avoid possible inter-experimental biases. Both reference and test aRNA were directly labeled using ULS aRNA Fluorescent labeling Kit (Kreatech, Netherlands) with Cy3 for reference and Cy5 for test samples.

Whole genome mouse 36 k oligo arrays were printed in the Infectious Disease and Immunogenetics Section of Transfusion Medicine (IDIS), Clinical Center, National Institute of Health, Bethesda using oligos purchased from Operon (Huntsville, Ala.). The Operon Array-Ready Oligo Set (AROS™) V 4.0 contains 35,852 longmer probes representing 25,000 genes and about 38,000 gene transcripts and also includes 380 controls. The design is based on the Ensemb1 Mouse Database release 26.33b.1, Mouse Genome Sequencing Project, NCBI RefSeq, Riken full-length cDNA clone sequence, and other GenBank sequence. The microarray is composed of 48 blocks and one spot is printed per probe per slide. Hybridization was carried out in a water bath at 42° C. for 18-24 hours and the arrays were then washed and scanned on a Gene Pix 4000 scanner at variable PMT to obtain optimized signal intensities with minimum (<1% spots) intensity saturation. Resulting data files were uploaded to the mAdb databank (http://nciarray.nci.nih.gov) and further analyzed using BRBArrayTools developed by the Biometric Research Branch, National Cancer Institute (30) (web site located at linus.nci.nih.gov/BRB-ArrayTools.html) and Cluster and Treeview software (31). The global gene-expression profiling consisted of 18 experimental samples. Subsequent filtering (80% gene presence across all experiments and at least 3-fold ratio change) selected 11,256 genes for further analysis. Gene ratios were average-corrected across experimental samples and displayed according to uncentered correlation algorithm (32).

### Statistical Analysis

Rate of tumor growth was compared statistically by un-paired Student's t test. Unsupervised analysis was performed for class confirmation using the BRBArrayTools and Stanford Cluster program (32). Class comparison was performed using parametric unpaired Student's t test or three-way ANOVA to identify differentially-expressed genes among tumor-bearing, tumor-rejection and relapse groups using different significance cut-off levels as demanded by the statistical power of each comparison. Statistical significance and adjustments for multiple test comparisons were based on univariate and multivariate permutation test as previously described (33, 34).

### Results

### T Cell-Mediated Rejection of MMC and Relapse of its Neu Antigen Negative Variant, ANV, in Wild-Type FVB Mouse

Wild-type FVB mice are capable of rejecting MMC within 3 weeks because of specific recognition of rat neu protein by their T cells as opposed to their transgenic counterparts, FVBN202, that tolerate rat neu protein and fail to reject MMC (21, 26). In order to determine whether aggressive rejection of primary MMC by T cells may lead to relapse-free survival in wild-type FVB mice, we performed follow-up studies. Animals (n=15) were challenged with MMC by subcutaneous (s.c.) inoculation at the right groin. Animals were then monitored for tumor growth twice weekly. All mice rejected MMC within 3 weeks after the challenge. However, a fraction of these animals (8 out of 15 mice) developed recurrent tumors at the site of inoculation. These relapsed tumors had lost neu expression under immune pressure (21, 26). Relapsed-free groups were maintained as breeding colonies and did not show any relapse during their life span. Splenocytes of FVB mice secreted IFN-γ in the presence of MMC only (2200 pg/ml) while no appreciable IFN-γ was detected when lymphocytes were stimulated with ANV (110 pg/ml). No IFN-γ was secreted by splenocytes or tumor cells alone (data not shown).

T Cells Derived from Wild-Type FVB Mice Will Induce Apoptosis in MMC

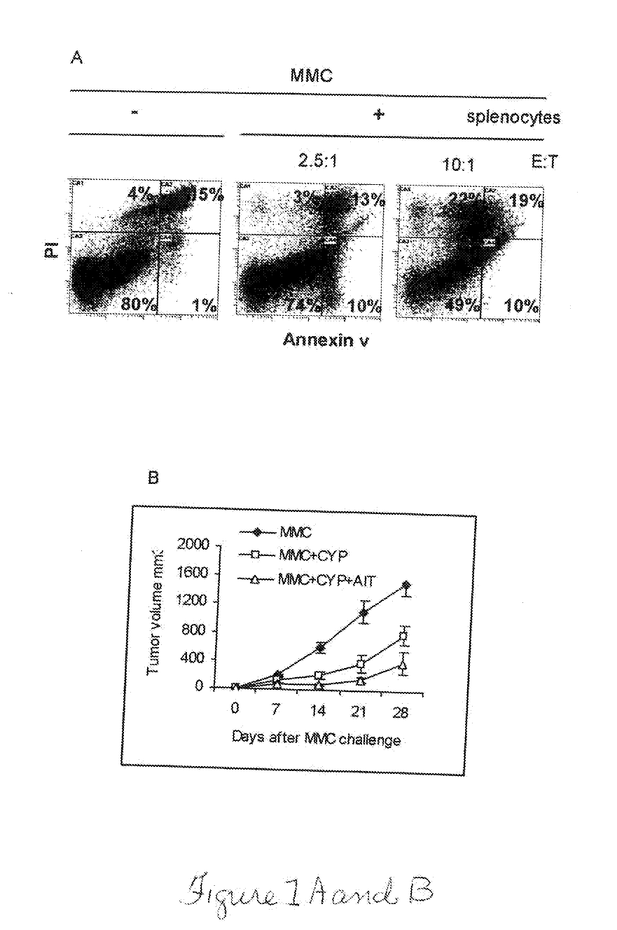

In order to determine whether neu-specific recognition of MMC by T cells may induce apoptosis in MMC, in vitro studies were performed. Splenocytes of naïve FVB mice were stimulated with irradiated MMC for 24 hrs followed by 3 day expansion in the presence of IL-2 (20 U/ml). Lymphocytes were then co-cultured with MMC (E:T ratio of 2.5:1 and 10:1) for 48 hrs in the presence of IL-2 (20 U/ml). Control wells were seeded with MMC or splenocytes alone in the presence of IL-2. Cells (floaters and adherents) were collected and subjected to a three color flow cytometry analysis using mouse anti-rat neu Ab (Ab-4), PE-conjugated anti mouse Ig, control Ig, annexin V, and PI. Gated neu positive cells were analyzed for the detection of annexin V+ and PI+ apoptotic cells. As shown in FIG. 1A, 80% of MMC were annexin V- and PI-in the absence of lymphocytes while only 49% of MMC were annexin V- and PI-in the presence of lymphocytes at 10:1 E:T ratio. At a lower E:T ratio (2.5:1) there was a slight dropping in the number of viable MMC (from 80% to 74%), but marked increase in the number of early apoptotic cells (annexin V+/PI−) from 1% to 10%. At a higher E:T ratio (10:1) early (Annexin V+/PI−) or late (annexin V+/F′1+) apoptotic cells and necrotic cells (annexin V-/P1+) were markedly increased.

Adoptive Immunotherapy (AIT) of FVBN202 Mice Using T Cells Derived from Wild-Type FVB Donors Failed to Reject MMC

In order to determine whether T cells Of FVB mice with neu-specific and anti-tumor activity may protect FVBN202 mice against MMC challenge, AIT was performed. Using nylon wool column, T cells were enriched from the spleen of FVB donor mice following the rejection of MMC. FVBN202 recipient mice were injected i.p. with cyclophosphamide (CYP; 100 μg/g) in order to deplete endogenous T cells. After 24 hrs animals were challenged with MMC tumors (4×10⁶ cells/mouse). Four-five hrs after tumor challenge, donor T cells were transferred into FVBN202 mice (6×10⁷ cells/mouse) by tail vein injections. Control FVBN202 mice were challenged with MMC in the presence or absence of CYP treatment. Animals were then monitored for tumor growth. As shown in FIG. 1B, CYP treatment of animals resulted in retardation of tumor growth in FVBN202 mice as expected. Student's t test analysis on days 14, 21, and 28 post-challenge showed significant differences between these two groups (P=0.005, 0.007, 0.01, respectively). Adoptive transfer of neu-specific effector T cells from MMC-sensitized FVB mice into CYP-treated FVBN202 groups did not significantly inhibit tumor growth compared to CYP-treated control groups (P>0.05). Adoptive transfer of neu-specific effector T cells from untreated FVB mice into CYP-treated FVBN202 groups showed similar trend of tumor growth (data not shown). These experiments suggest that T cell responses associated with MMC rejection in wild-type FVB mice (21) may represent an epiphenomenon with no true cause-effect relationship or that FVBN202 mice retain tolerogenic properties in spite of CYP treatment that can hamper the function of potentially effective anti-cancer T cell responses. We favor the second hypothesis based on our previous depletion experiments that demonstrated the requirement of endogenous effector T cells of FVB mice for rejection of MMC tumors (21).

### Genetic Signatures Defining Rejection or Tolerance of MMC Tumors

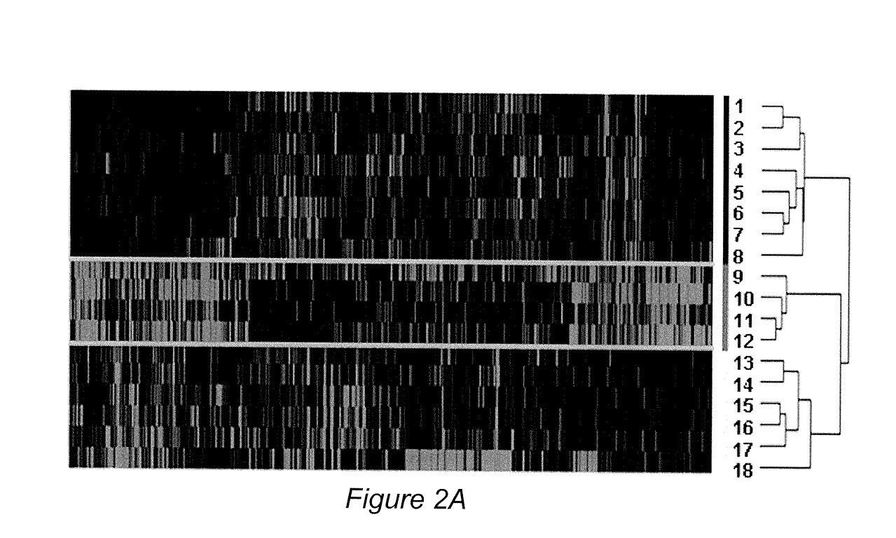

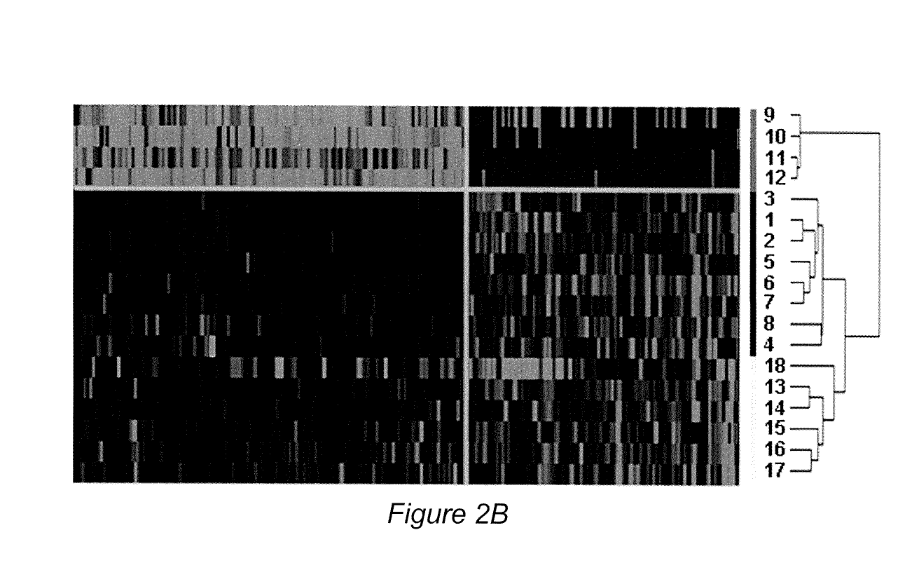

To ascertain whether the presence of neu-specific effector T cells may trigger a cascade of events which may determine success or failure in tumor rejection, wild-type FVB and FVBN202 mice were inoculated with MMC. Historically, all FVB mice reject MMC, however a fraction develop a latent tumor relapse. In contrast, FVBN202 mice fail to reject transplanted MMC. Ten days after the tumor challenge, transplanted MMC tumors were excised and RNAs were extracted from both FVB and FVBN202 carrier mice based on the presumption that the biology of the former would be representative of active tumor rejection and that of the latter representative of tumor tolerance. Thus, the timing of tumor harvest was chosen to capture transcriptional signatures associated with the active phase of the tumor rejection process in wild-type FVB mice in comparison with the corresponding tolerance of spontaneous mammary tumors in the FVBN202 mice. We speculated that this comparison would allow distinguishing whether tolerance was due to inhibition of T cell function within the tumor microenvironment of spontaneous mammary tumors or to a complete absence of such responses. To enhance the robustness of the comparison, a similar analysis was performed extracting total RNA from spontaneous tumor in FVBN202 mice. In addition, RNA was extracted from MMC tumors in wild-type FVB mice that experienced tumor recurrence following the initial rejection of MMC. This second analysis allowed the comparison of mechanisms of tumor evasion in the absence of known tolerogenic effects. Micro array analyses were then performed on the amplified RNA (aRNA) extracted from these tumors using 36 k oligo mouse arrays. Hence, genes considered as differentially expressed in the study groups could either represent MMC tumor cells or host cells infiltrating the tumor site. Probes with missing values greater than 80% or a change less than 3 fold were excluded from further analysis. Unsupervised clustering demonstrated outstanding differences among the three experimental groups (FIG. 2A). Genes of spontaneous mammary tumors (samples 13, 15, 16, 17) clustered closely to those of transplanted MMC (14 and 19) excised from FVBN202 mice suggesting that the biology of MMC tumors remains comparable between these two experimental models of tolerance. Global transcriptional patterns associated with tumor relapse (samples 1-8) were instead clearly different from those of spontaneous mammary tumors or MMC transplanted in tolerant FVBN202 mice suggesting that a completely different biological process was at the basis of tumor evasion through loss of target antigen expression. Finally, MMC tumors undergoing rejection (samples 9-12) were clearly separated from the either kind of non-regressing tumors.

### Biomarkers of Rejection

Our first class comparison searched for differences between the four tumor samples undergoing rejection and the rest of the MMC tumors whether belonging to the tolerogenic or the evasion process. This approach followed the exclusion principle whereby factors determining the occurrence of a phenomenon should be discernible from unrelated ones independent of the causes preventing its occurrence. An unpaired Student t test with a cut-off set at p<0.001 identified 2,449 genes differentially expressed between regressing and non regressing tumors (permutation p value=0) of which 1,003 genes were upregulated in regressing tumors clearly distinguishing the two categories (FIG. 2B). Of those, a large number were associated with immune regulatory functions. Gene Ontology databank was queried to assign genes to functional categories and upregulated pathways were ranked according to the number of genes identified by the study belonging to each category (FIG. 2C). The top categories of genes that were upregulated in primary rejected MMC tumors were Cytokine-Cytokine interaction, MAPK signaling, Cell Adhesion related transcripts and axon guidance, T cell receptor, JAK-STAT and Toll-like receptor signaling pathways. NK cell mediated cytotoxicity and calcium signaling pathways were also enriched in upregulated genes. In contrast, very little evidence of immune activation could be observed in either category of non-regressing tumors suggesting that lack of immune rejection is due to absent or severely hampered immune responses in the tumor microenvironment independent of the mechanisms leading to this resistance.

To better describe the immununological pathways associated with tumor regression we organized genes with immune function into three categories including chemokines, IFN-α2, IFN-γ and interferon-stimulated genes (ISGs) (Table 1, presented in FIG. 4A), and cytokines and signaling molecules (Table 2, presented in FIG. 4B). From this analysis, it became clear that T cell infiltration into tumors was associated with activation of various pathways leading to the expression of IFN-α, IFN-γ and several ISGs including interferon regulatory factor (IRF)-4, IRF-6 and STAT-2. In addition, several cytotoxic molecules were overexpressed including calgranulin-a, calgranulin-b and granzyme-B; all of them representing classical markers of effector T cell activation in humans (10) and in mice (35). Thus, tumor rejection in this model clearly recapitulates patterns observed in various human studies in which expression of ISGs is associated with the activation of cytotoxic mechanisms among which granzyme-B appears to play a central role.

Is there a Difference Between Signatures of Immune Evasion and Immune Tolerance?

As shown in Table 3 (presented in FIG. 4C), the high expression of IL-10 and the IL-10 receptor-β chain concordant with IRF-1 in the tolerogenic model strongly suggests the presence of regulatory mechanism within the microenvironment of MCC-bearing FVBN202 mice. Preferential expression of SOCS-1 and SOCS-3 in the microenvironment of MMC tumors of FVBN202 mice also strongly suggest a marked activation of regulatory functions present in the tolerized host (Table 3, presented in FIG. 4C).

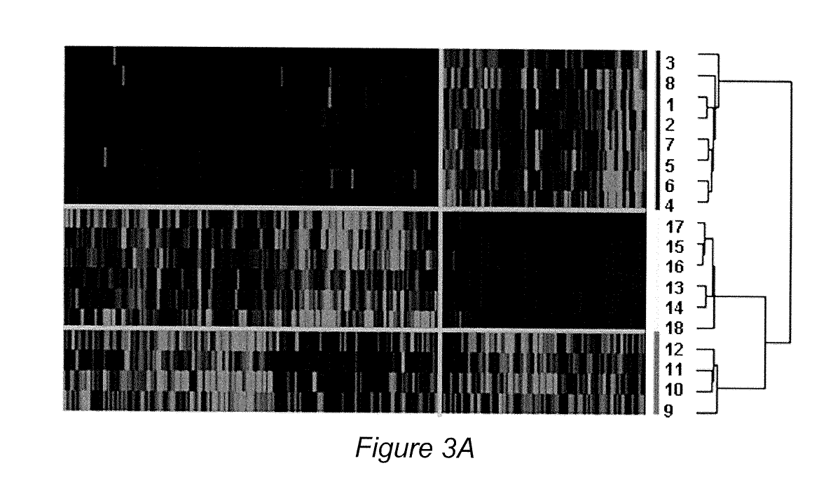

To further investigate whether similar mechanisms were involved in failure of tumor rejection in the tolerance model and the evasion model, we characterized potential differences between the two models of immune resistance; we compared statistical differences between the tolerogenic and the evasion model comparing the two non-regressing groups by unpaired Student t test using as a significance threshold a p-value <0.001. This analysis was performed on pre-selected genes that had been filtered for an at least 80% presence of data in the whole data set and a minimal fold increase of 3 in at least one experiment (FIG. 3). This analysis identified 1,369 genes differentially expressed by the two groups (multivariate permutation test p-value=0) of which 462 were upregulated in the tolerogenic model and 907 were upregulated in the tumor evasion model (FIG. 3A). Several of these genes where specifically expressed by either group although the expression of a few of them was shared by the regressing MCC tumors. Annotations and functional analysis based on Genontology data base (GEO) demonstrated that the predominant functional classes of genes transcriptionally active in one of the other type of non-responding MMC tumors were not associated with classical activation of T cell effector functions but rather were associated with more general metabolic processes (FIGS. 3B and 3C). However, detailed analysis of transcripts associated with immunological function (Table 3, presented in FIG. 4C) defined dramatic differences between the two mechanisms of immune resistance.

### Discussion

FVB mice reject primary MMC by T cell-mediated neu-specific immune responses. However, a fraction of animals develop tumor relapse after a long latency. On the other hand their transgenic counterparts, FVBN202, fail to mount effective neu-specific immune responses and develop tumors (21). Although FVBN202 mice appear to elicit weak immune responses against the neu protein within a certain window of time (24), the neu expressing MMC tumors are still well tolerated and animals develop spontaneous mammary tumors. Despite the observation that T cells derived from FVB mice were capable of recognizing MMC and inducing apoptosis in these tumors in vitro, adoptive transfer of such effector T cells into FVBN202 mice failed to protect these animals against challenge with MMC.

It has been suggested that T cells play a significant role in determining the natural history of colon (14-16) and ovarian (17) cancer in humans. Transcriptional signatures have been identified that suggest not only T cell localization but also activation through the expression of IFN-γ, ISGs and cytotoxic effector molecules such as granzyme-B (10). We have recently shown that rejection of basal cell cancer induced by the activation of Toll-like receptor agonists also is mediated, at least in part, by localization and activation of CD8 expressing T cells with increased expression of cytotoxic molecules (36). Yet, a comprehensive experimental overview of the biological process associated with tumor rejection in its active phase has not been reported. Thus, our first class comparison searched for differences between the four tumor samples undergoing rejection and the rest of the MMC tumors whether belonging to the tolerogenic or the evasion process. Unlike non-regressing tumors (tolerance and evasion models), regressing tumors (rejection model) showed upregulation of immune activation genes, suggesting that failure in tumor rejection is due to immune evasion or severely hampered immune responses in the tumor microenvironment. A particularly interesting observation was the relative low expression of ISGs, with the exception of IRF-2 bp1. While the transcriptional patterns differentiating regressing from non-regressing tumors were striking and in many ways representative of previous observations in humans by our and others groups (37), differences among MMC tumors non-regressing in FVBN202 mice and those relapsing after regression in FVB mice were subtle. We have previously proposed that lack of regression of human tumors is primarily associated with indolent immune responses rather than dramatic changes in the tumor microenvironment enacted to counterbalance a powerful effector immune response (13, 33, 37). The MMC tolerance model allowed investigating this hypothesis at least in this restricted case. Spontaneous mammary tumors or transplanted MMC tumors in FVBN202 mice displayed immune suppressive properties that were identified by transcriptional profiling through the activation of genes associated with regulatory function. This would occur only in case an indolent adaptive immune response occurred in these transgenic mice and was hampered at the tumor site by a mechanism of peripheral suppression. If however, central tolerance was the reason for the lack of rejection, minimal changes should be observed in tolerogenic model similar to those detectable in the tumor evasion model where MMC tumors lost expression of HER-2/neu and become irrelevant targets for HER-2/neu-specific T cell responses. The presence of regulatory mechanism within the microenvironment of MCC-bearing FVBN202 mice was associated with increased IL-10 as well as increased expression of SOCS-1 and SOCS-3. It has recently been shown that myeloid-derived suppressor cells (MDCS) induce macrophages to secret IL-10 and suppress anti-tumor immune responses (38). Importantly, it was shown that high levels of MDCS in neu transgenic mice would suppress anti-tumor immune responses against tumors (39). Interleukin-10 is increasingly recognized to be strongly associated with regulatory T cell (40) and M2 type tumor-associated macrophage function (41) and its expression is mediated in the context of chronic inflammatory stimuli by the over-expression of IRF-1. SOCS-1 inhibits type I IFN response, CD40 expression in macrophages, and TLR signaling (42-44). Expression of SOCS-3 in DCs converts them into tolerogenic DCs and support Th-2 differentiation (45). Importantly, tumors that express SOCS-3 show IFN-γ resistance (46). Unlike tolerance model, recurrence model revealed expression of Igtp, suggesting the involvement of IFN-γ in this model (Table 3). This observation is consistent with our previous findings on the role of IFN-γ in neu loss and tumor recurrence (21).

MMC tumors evading immune recognition had undergone a process of complex immune editing that resulted not only in the loss of the HER-2/neu target antigen but also in the upregulation of various Th2 type cytokines such as IL-4 and, IL-13 (47) and the corresponding transcription factor IRF-7 over-expression predominantly associated to a deviation from cellular Th-1 to Th-2 and humoral type immune responses (48). In addition, the microenvironment of recurrent tumors was characterized by the coordinate expression of STAT-4, IL12b, IL-23r and IL-17; this cascade has been associated with the development of Th17 type immune responses that play a dominant role in autoimmune inflammation (49, 50) and T-cell dependent cancer rejection (51, 52). Since both humoral and cellular immune responses are potentially involved in the rejection of HER-2/neu expressing tumors (53), this data suggests that a cognitive and active immune response is still attempting to eradicate MMC tumors which may still express subliminal levels of the target antigen. However, the overall balance between host and cancer cells favors, in the end, tumor cell growth because the expression of HER-2/neu, the primary target of both cellular and humoral responses, is critically reduced.

Altogether, these observations suggest that neu antigen loss and subsequent immunological evasion from cellular Th-1 to Th-2 and humoral type immune response is a major mechanism in evasion model while peripheral suppression such as sustained IL-10, SOCS I and SOCS3 expression is a major player in tolerance model. This conclusion provides a satisfactory explanation for the lack of rejection of MMC tumors in FVBN202 mice receiving adoptively transferred HER-2/neu-specific T cells. In this case, effective T cell responses exclude central tolerance or peripheral ignorance as the only mechanism potentially hampering their effector function at the tumor site suggesting that other regulatory mechanisms such as peripheral suppression could be responsible for inactivation of donor effector T cells. High levels of MDSC in neu transgenic mice support this possibility, and the role and mechanisms of MDSC in suppression of adoptively transferred neu-specific T cells remain to be determined in FVBN202 mice.

### Example 2

### Immune Function Gene Signatures in Human Breast Cancer

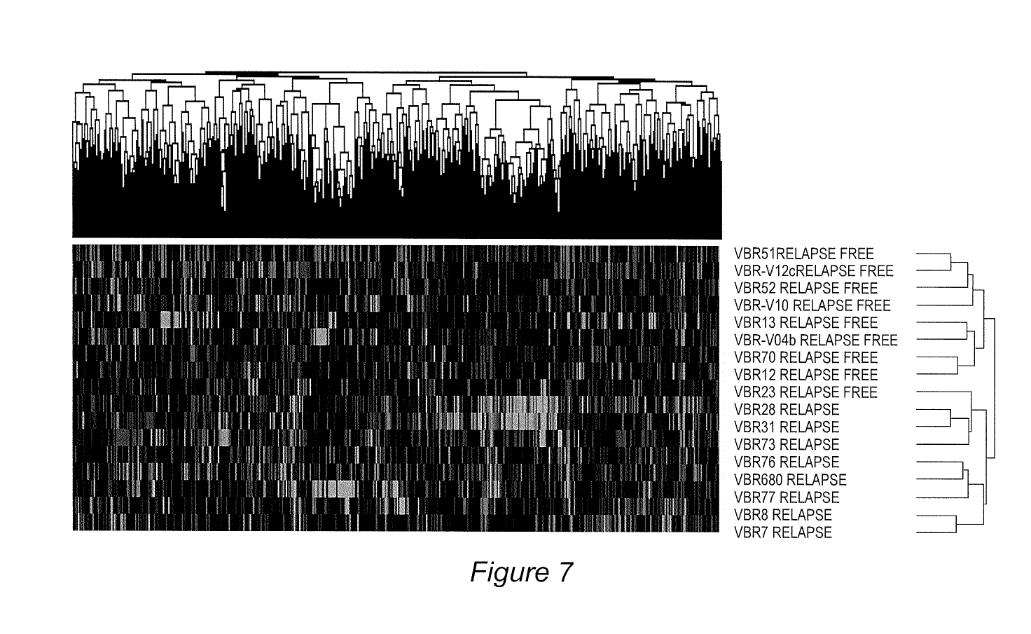

In order to validate signatures of immune function genes that had been detected in the animal (mouse) model of breast carcinoma, gene expression (RNA) in tumor lesions of human breast cancer patients who either remained relapse-free or developed relapse within 1-5 years after the initial treatment was analyzed. As shown in FIG. 7, unsupervised analysis of gene arrays from breast cancer patients compared to reference (control) human peripheral blood mononuclear cells (PBMCs) showed that relapse-free and relapse patients exhibit different, distinct clusterings of expressed genes. 9,797 genes were identified which exhibited at least a 3-fold change in expression between relapsed and relapse-free individuals.

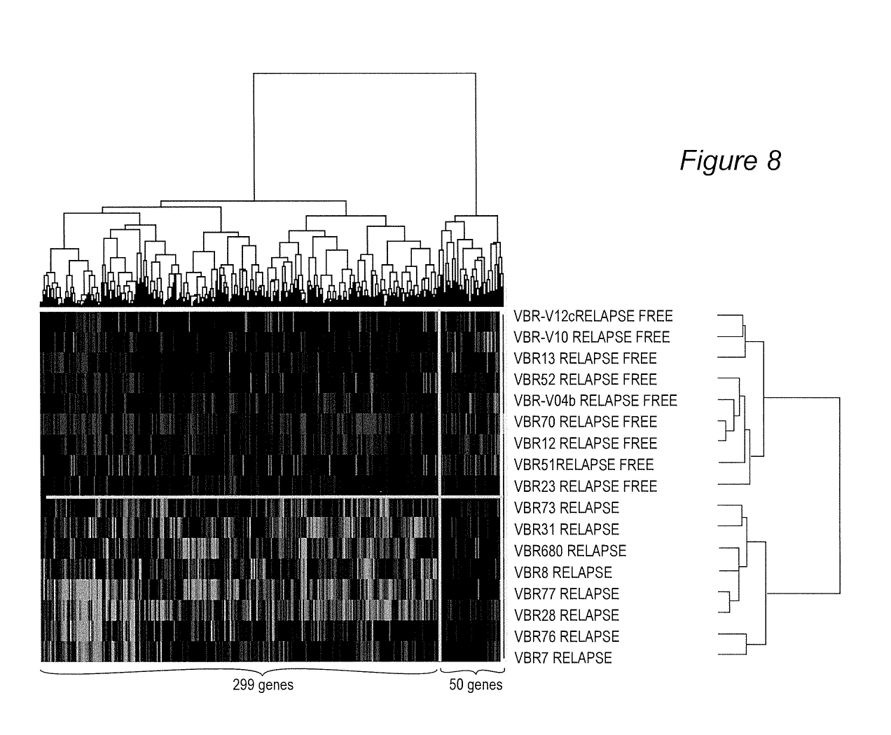

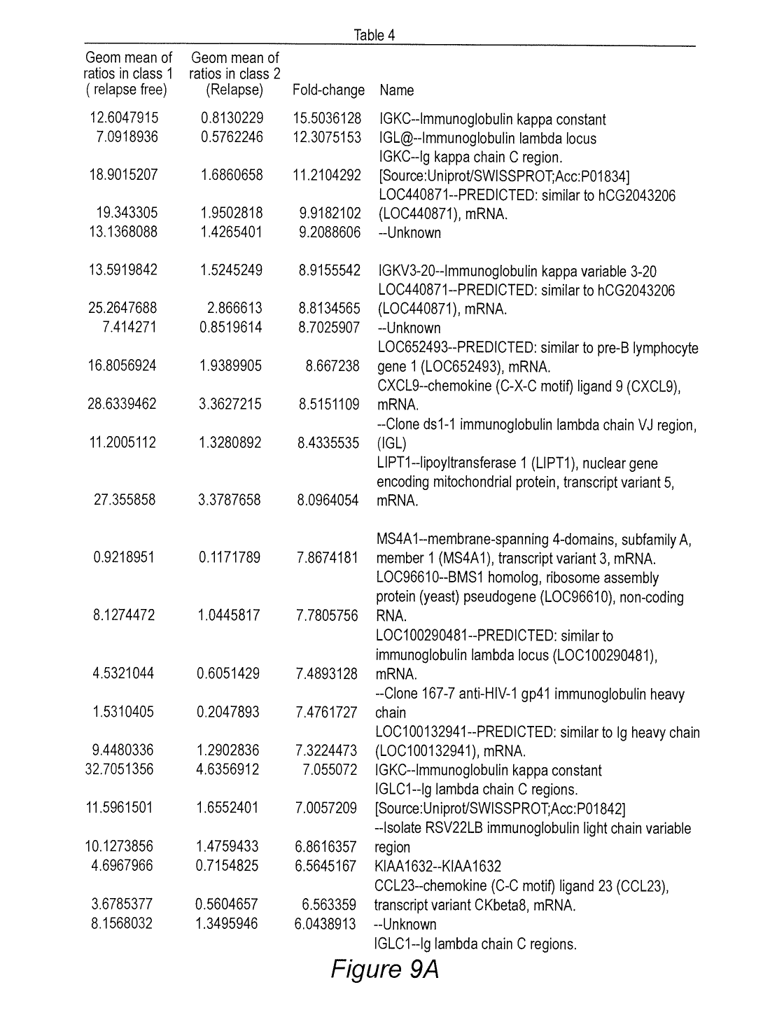

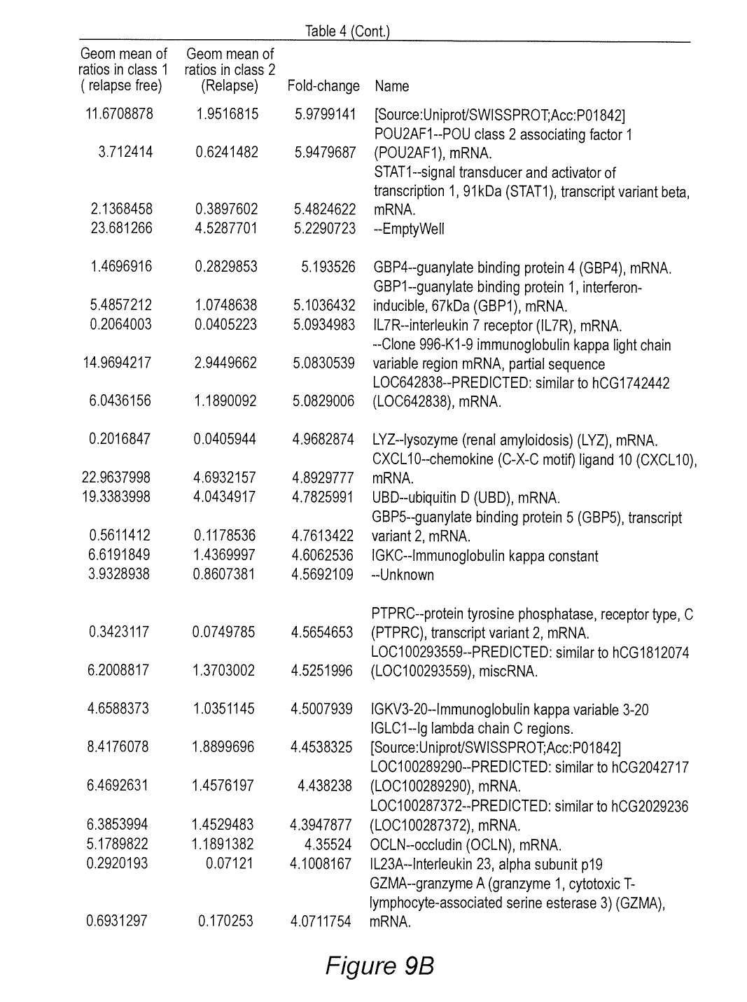

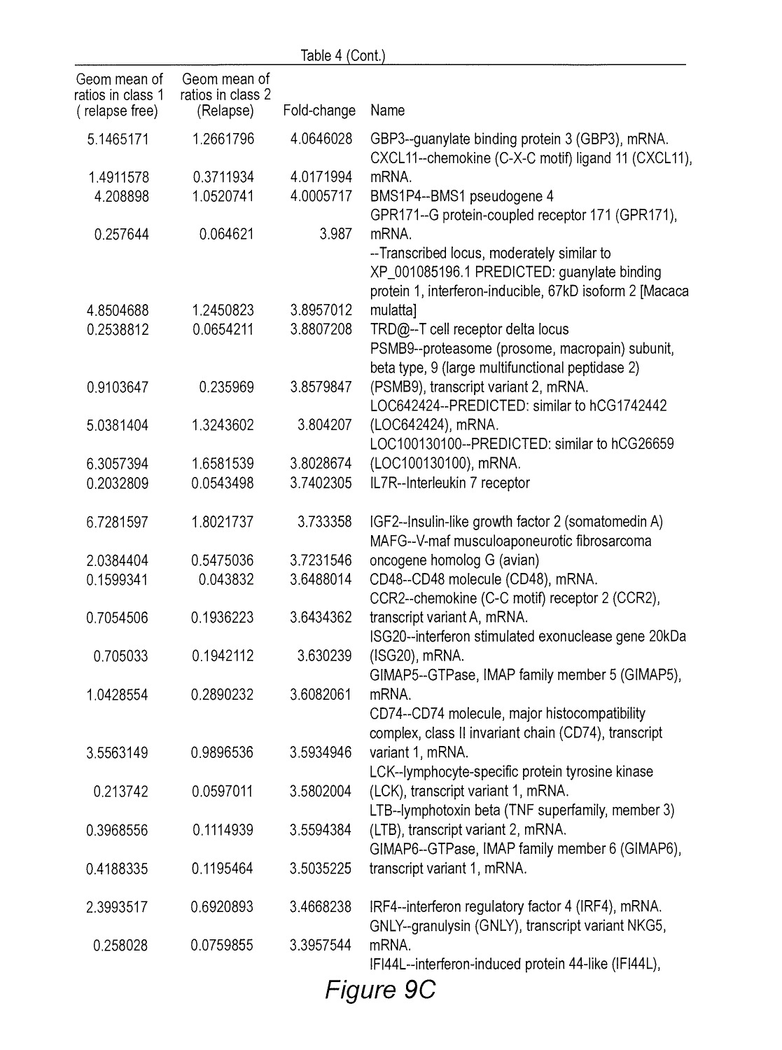

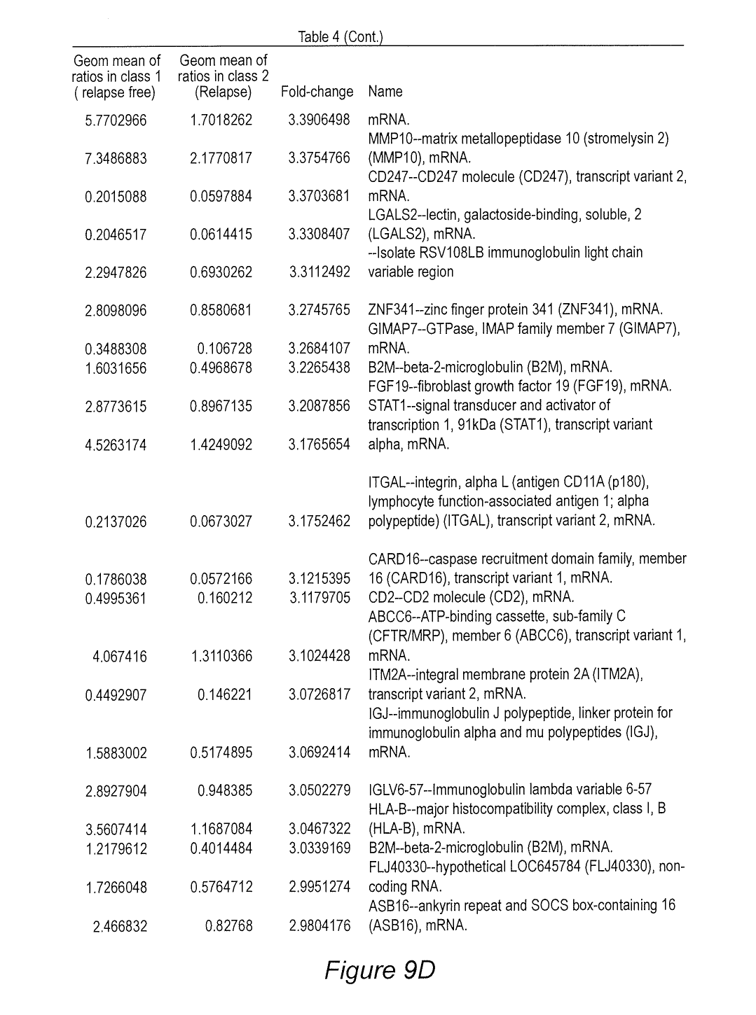

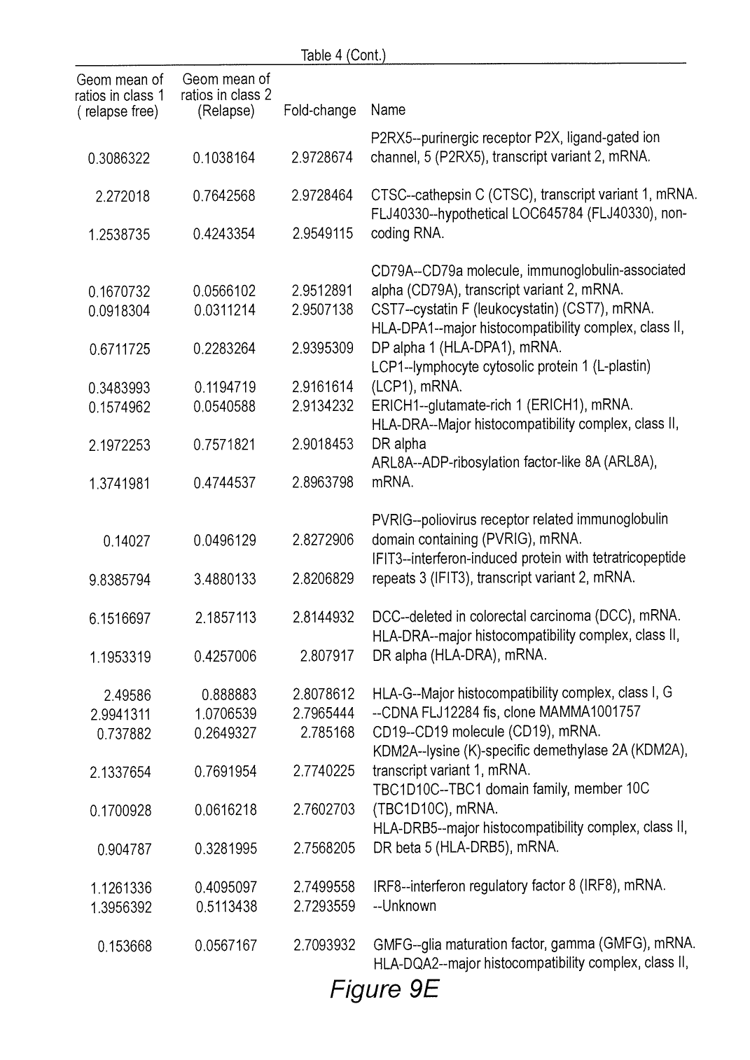

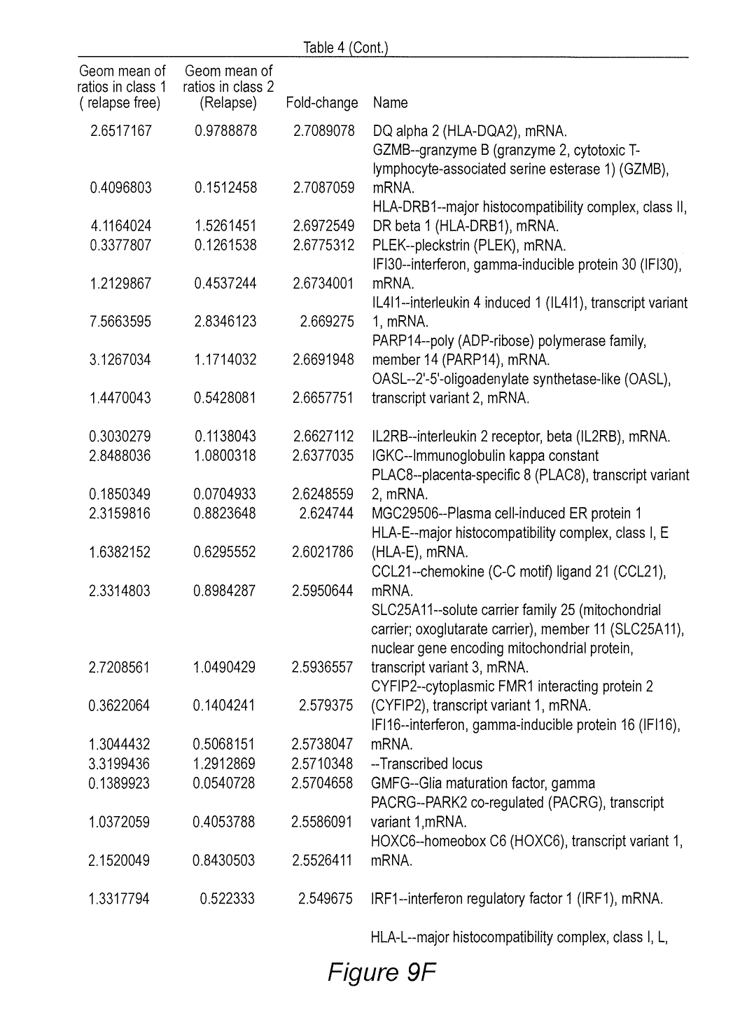

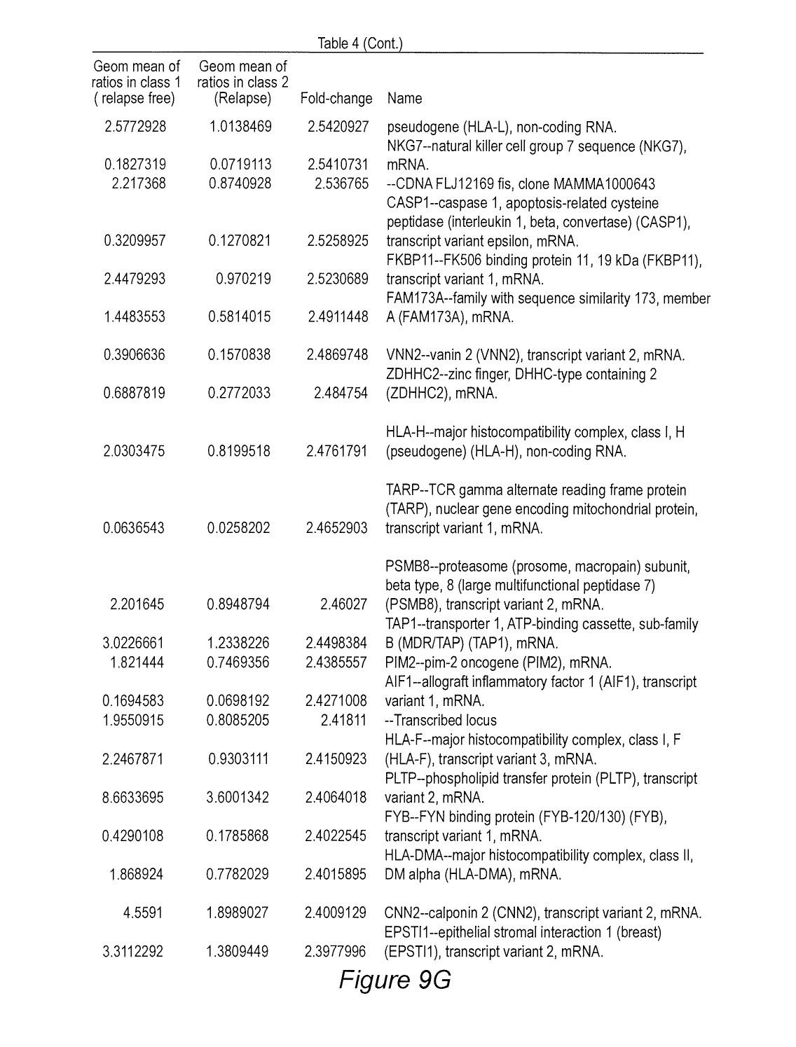

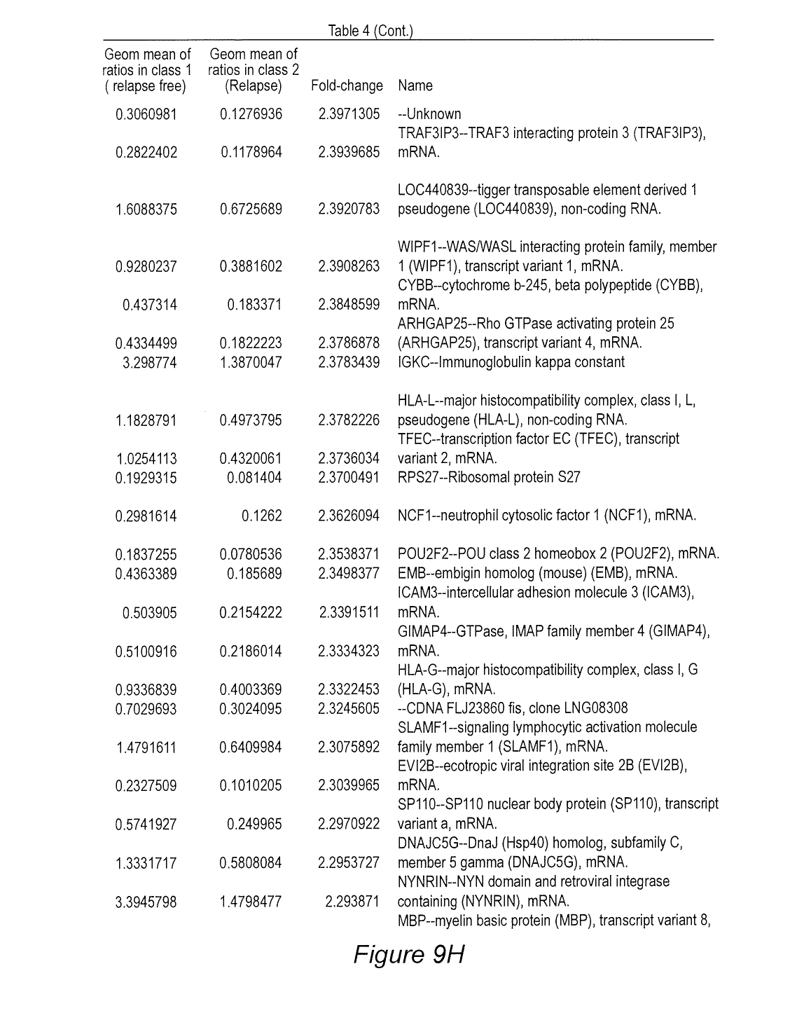

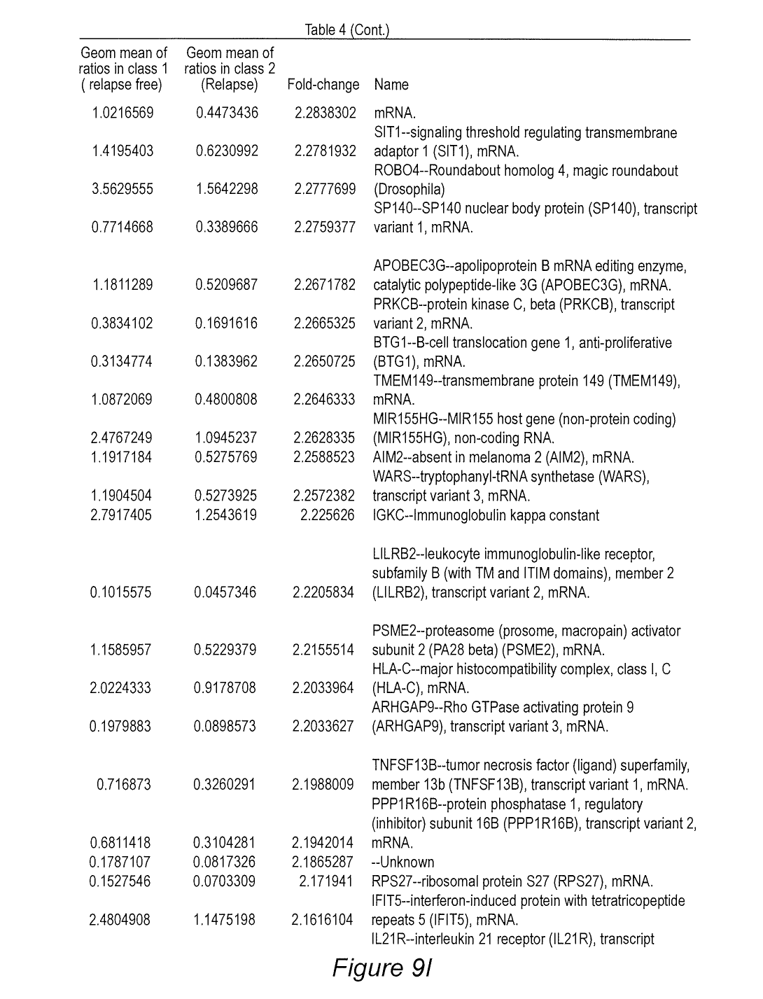

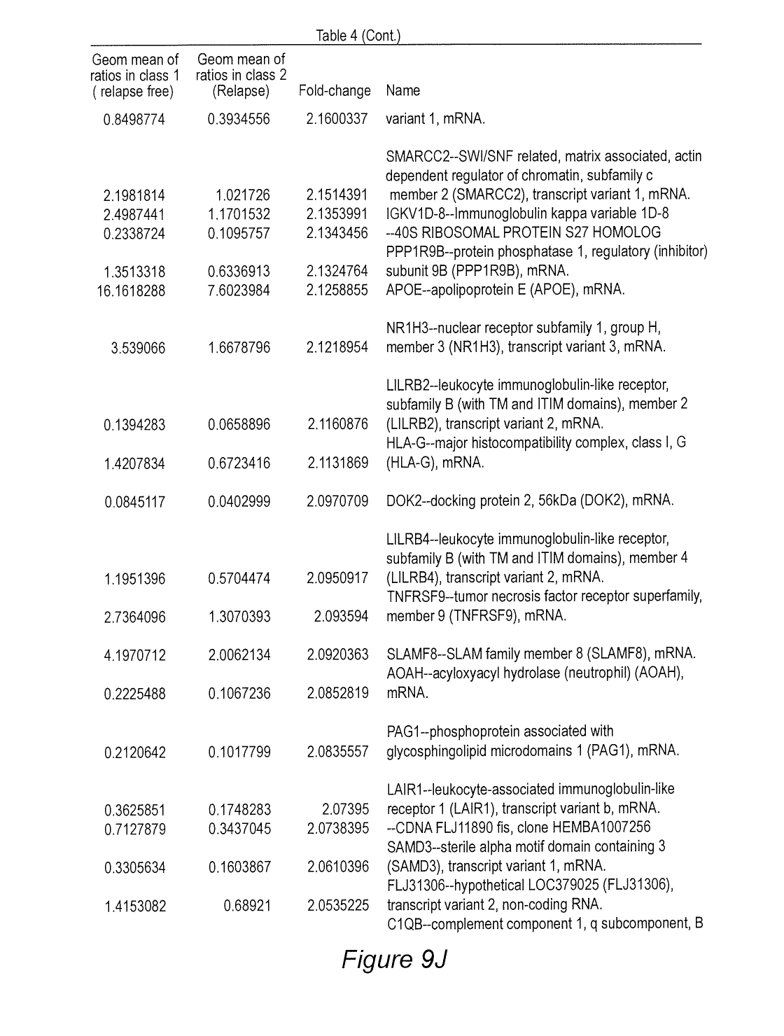

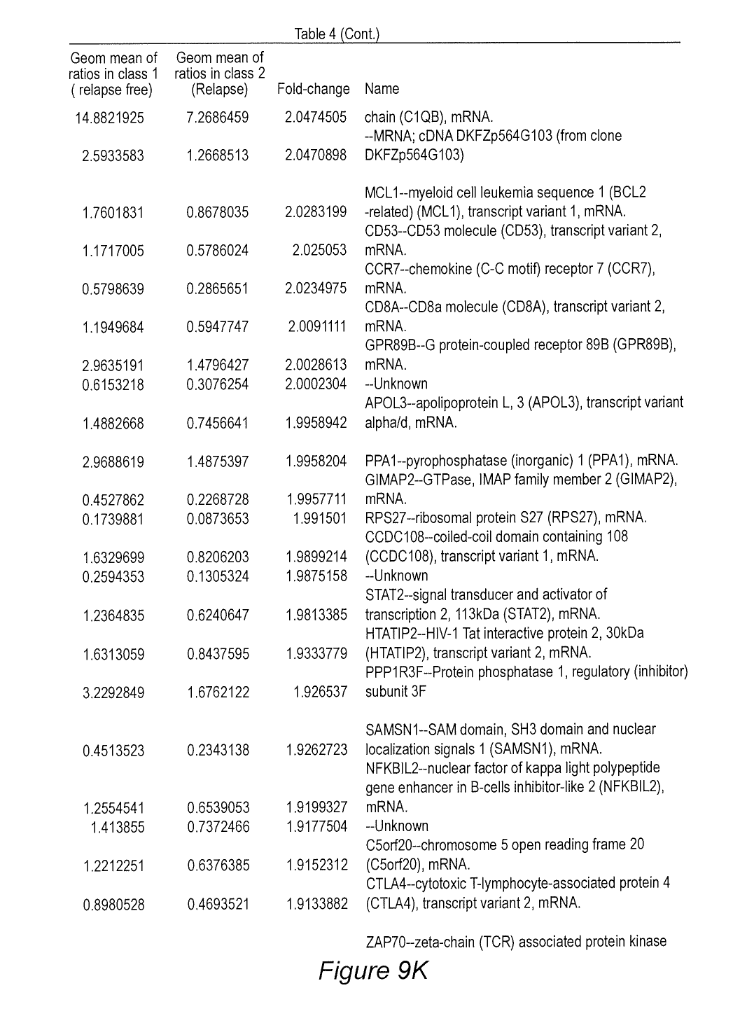

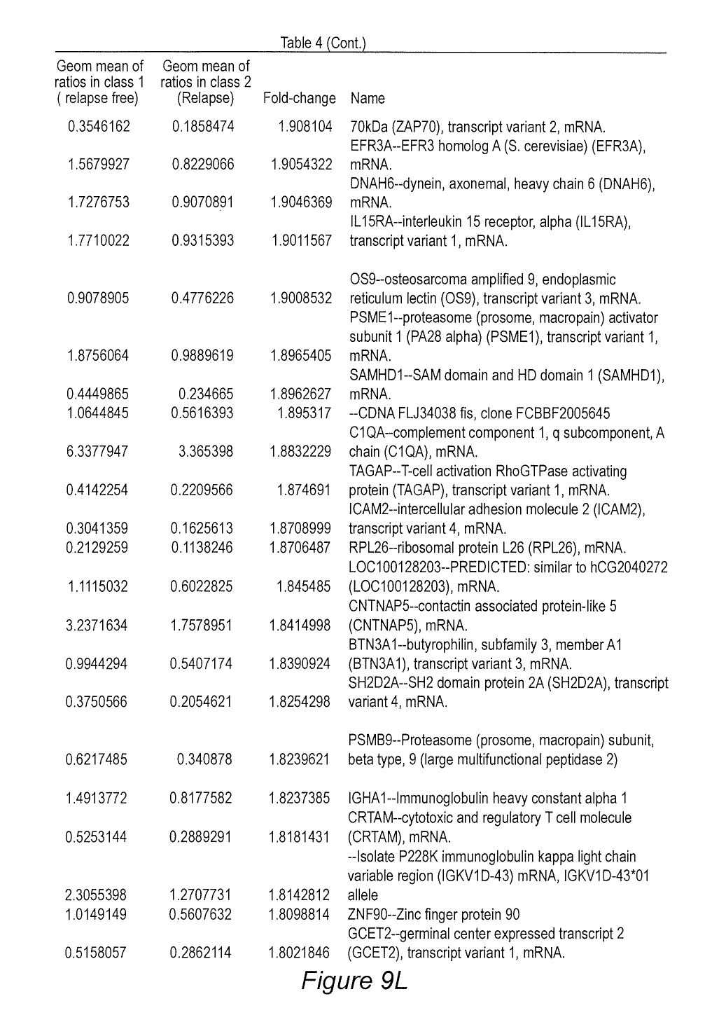

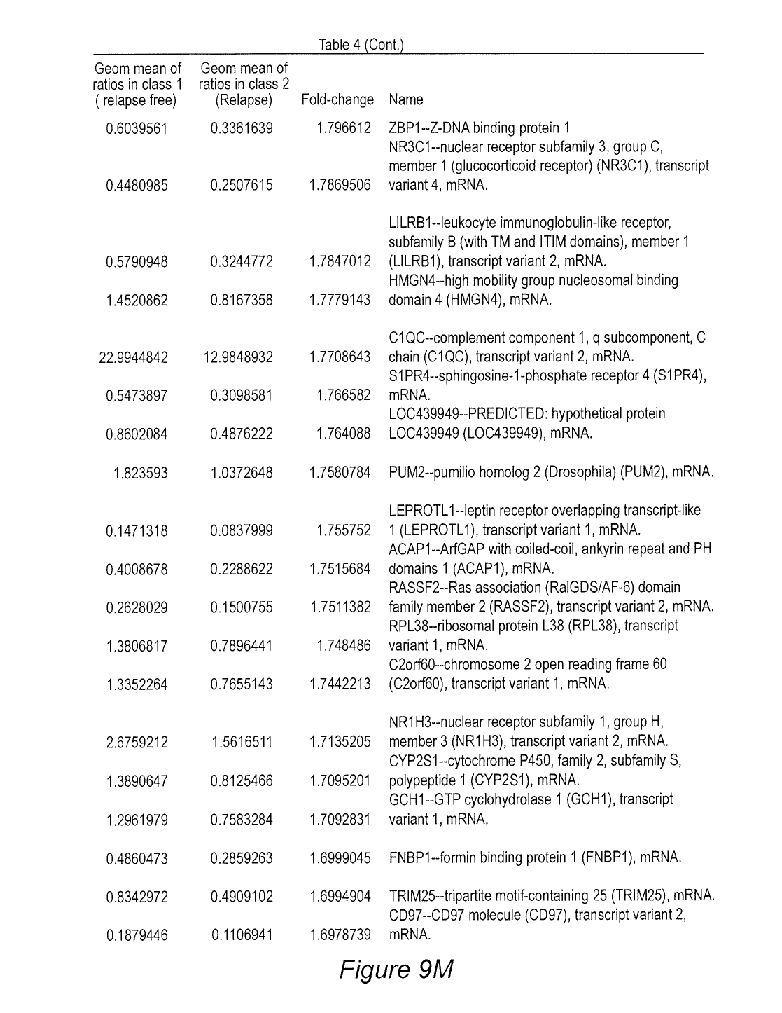

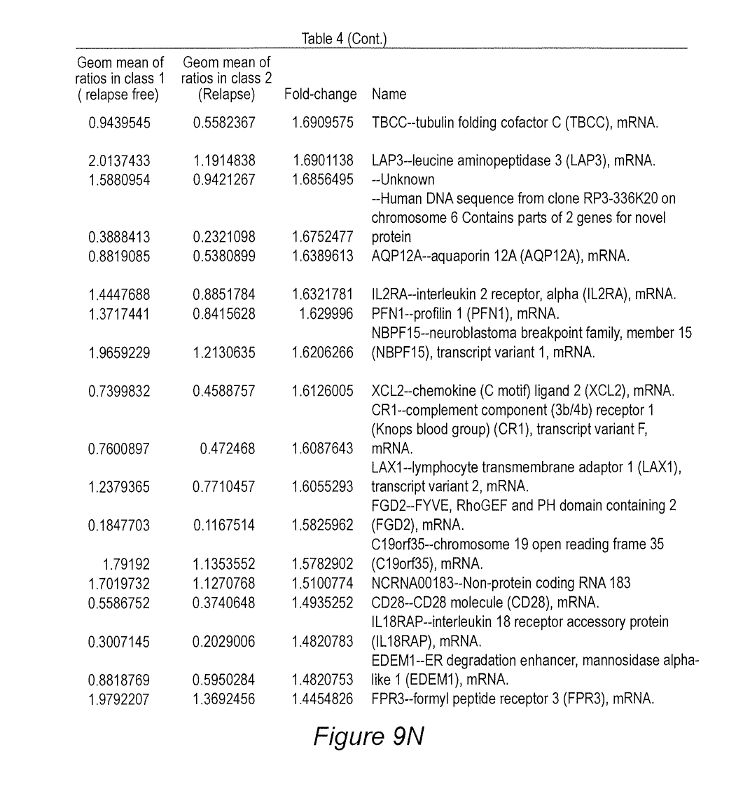

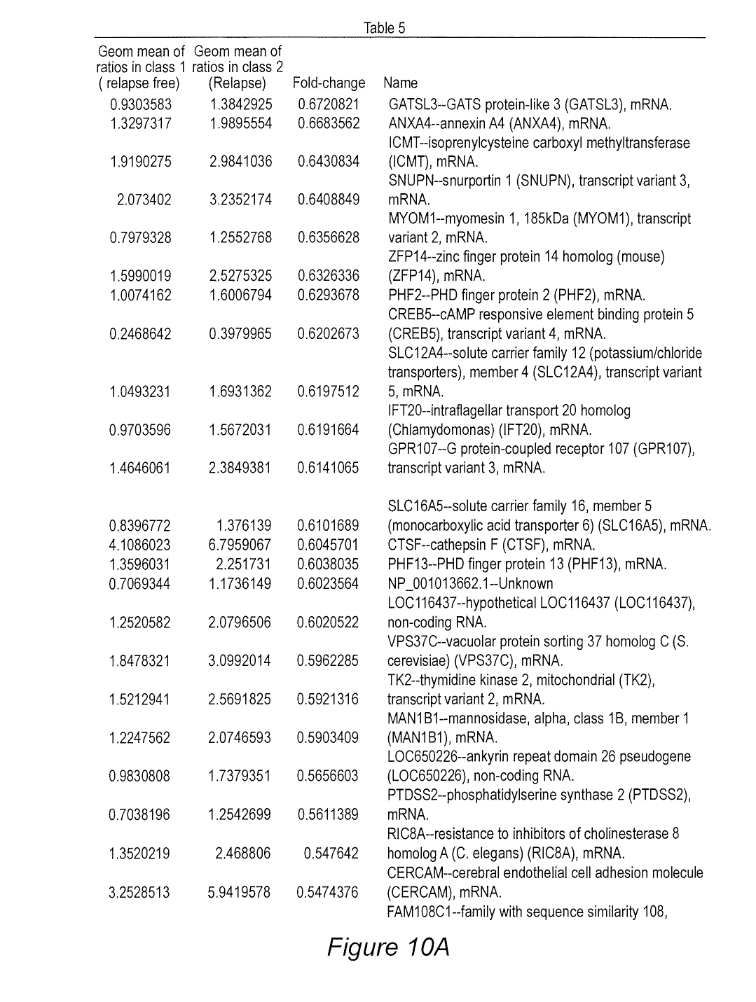

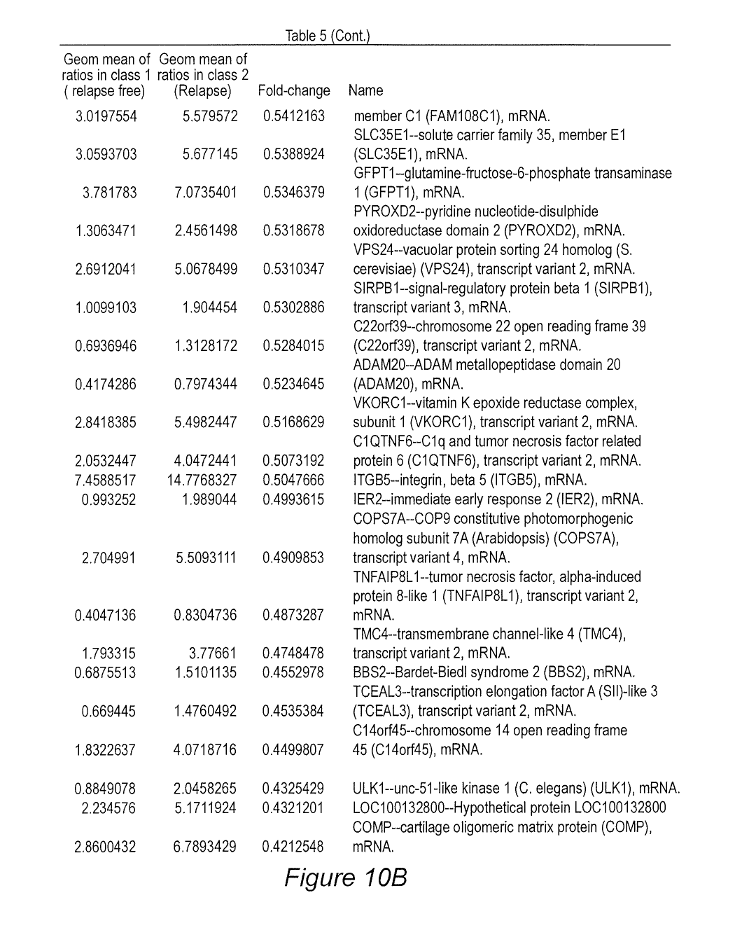

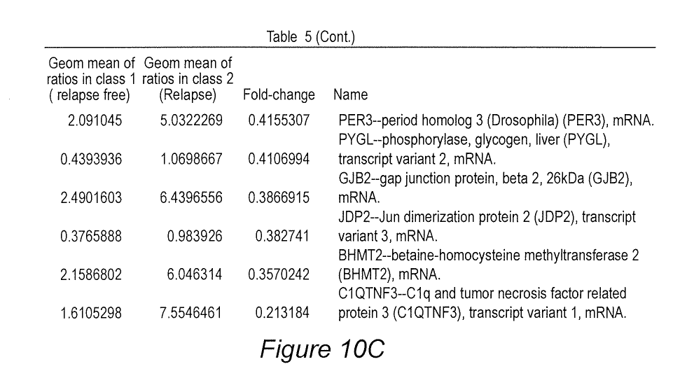

Supervised analysis at p value<0.001 revealed upregulation of 50 genes in patients with relapse as well as upregulation of 299 genes in patients with relapse-free survival (FIG. 8). These genes are listed in tabular form in Figures Tables 4 and 5, respectively. Table 4 is presented in FIGS. 9A-L and Table 5 is presented in FIGS. 10A and B. Further canonical pathway analysis of immune function genes revealed that relapse-free survival is associated with upregulation of a network of genes related to B cell development, antigen presentation, graft vs host disease signaling, interferon signaling and primary immunodeficiency signaling (FIGS. 12A-E). In addition, representative genes involved in these pathways were further validated by detecting protein products using IHC where commercial antibodies were available (data not shown).

Novel findings resulting from this work include the following: 1) one single gene/cell of the immune response cannot predict the outcome with respect to relapse; rather, a network (pattern, signature, etc.) of immune cell activation is required for prognosis; and 2) upregulation of genes involved in immune suppression along with upregulation of genes that are involved in host protection can explain why patients with an intact immune system can still develop cancer. Conventional cancer therapy would change this balance in favor of immune effector signature because genes involved in immune suppression (such as NO) are upregulated along with immune effector genes (listed in FIG. 11) and are usually induced because of the presence of the tumors.

In summary, 50 genes associated with tumor relapse and 299 genes associated with relapse-free survival have been identified. Expression patterns of these disease-outcome standard genes can be used as references to analyze gene expression in tumor samples obtained from cancer patients, and to determine or predict the likely prognosis for the patient, thereby influencing the choice of treatments for the patient.

### Example 3

### Clinical Applications

During a routine mammogram or during routine self-examination, a “lump” is discovered in the breast tissue of a human patient. Biopsy samples are taken from the lump and from the surrounding tissue and it is determined that the patient has breast cancer. In addition to routine biopsy analysis, the samples are assessed using the methods of the invention to determine whether the patient is likely to relapse after treatment or is likely to be relapse free after treatment, as follows:

The results provided by the methods of the invention generally lead to two possible outcomes:

Outcome 1: Analysis of the gene expression patterns in the microenvironment of the tumor reveal a pattern of gene expression that is biased toward upregulation of the 299 genes listed in Table 4. As a result of this finding, the patient's health care team concludes that the prognosis for the patient (e.g. after removal of the tumor) is favorable, and that recurrence (relapse) is unlikely. Recommended treatment includes, for example, vaccination and/or drugs that boost the immune system such as revlimid. But the patient may be spared the inconvenience and discomfort of more aggressive therapy.
Outcome 2: Analysis of the gene expression patterns in the microenvironment of the tumor reveal a pattern of gene expression that is biased toward upregulation of the 30 genes listed in Table 5. As a result of this finding, the patient's health care team concludes that the prognosis for the patient (e.g. after removal of the tumor) is not favorable, and that recurrence is likely. Recommended treatment includes: extensive surgery to remove surrounding tissues, using an aggressive chemotherapeutic regimen (e.g. drugs such as fludarabine, cyclophosphamide, IFN-γ, fludarabine, cyclophosphamide, amd gemcitabine) and aggressive radiation therapies.

### REFERENCES

1. Marincola F M, Rivoltini L, Salgaller M L, Player M, Rosenberg S A. Differential anti-MART-1/MelanA CTL activity in peripheral blood of HLA-A2 melanoma patients in comparison to healthy donors: evidence for in vivo priming by tumor cells. J Immunother 1996; 19:266-77.
2. D'Souza S, Rimoldi D, Lienard D, Lejeune F, Cerottini J C, Romero P. Circulating Melan-A/Mart-1 specific cytolytic T lymphocyte precursors in HLA-A2+ melanoma patients have a memory phenotype. Int J Cancer 1998; 78:699-706.
3. Lee K-H, Wang E, Nielsen M-B, et al. Increased vaccine-specific T cell frequency after peptide-based vaccination correlates with increased susceptibility to in vitro stimulation but does not lead to tumor regression. J Immunol 1999; 163:6292-300.
4. Marincola F M. A balanced review of the status of T cell-based therapy against cancer. J Transl Med 2005; 3:16.
5. Panelli M C, Riker A, Kammula U S, et al. Expansion of Tumor-T cell pairs from Fine Needle Aspirates of Melanoma Metastases. J Immunol 2000; 164:495-504.
6. Zippelius A, Batard P, Rubio-Godoy V, et al. Effector function of human tumor-specific CD8 T cells in melanoma lesions: a state of local functional tolerance. Cancer Res 2004; 64:2865-73.
7. Rosenberg S A, Sherry R M, Morton K E, et al. Tumor progression can occur despite the induction of very high levels of self/tumor antigen-specific CD8+ T cells in patients with melanoma. J Immunol 2005; 175:6169-76.
8. Disis M L, Calenoff E, McLaughlin G, et al. Existent T-cell and antibody immunity to HER-2/neu protein in patients with breast cancer. Cancer Res 1994; 54:16-20.
9. Lee P P, Yee C, Savage P A, et al. Characterization of circulating T cells specific for tumor-associated antigens in melanoma patients. Nat. Med 1999; 5:677-85.
10. Monsurro' V, Wang E, Yamano Y, et al. Quiescent phenotype of tumor-specific CD8+ T cells following immunization. Blood 2004; 104:1970-8.
11. Nagorsen D, Voigt S, Berg E, Stein H, Thiel E, Loddenkemper C. Tumor-infiltrating macrophages and dendritic cells in human colorectal cancer: relation to local regulatory T cells, systemic T-cell response against tumor-associated antigens and survival. J Transl Med 2007; 5:62.
12. Marincola F M, Jaffe E M, Hicklin D J, Ferrone S. Escape of human solid tumors from T cell recognition: molecular mechanisms and functional significance. Adv Immunol 2000; 74:181-273.
13. Marincola F M, Wang E, Herlyn M, Seliger B, Ferrone S. Tumors as elusive targets of T cell-based active immunotherapy. Trends Immunol 2003; 24:335-42.
14. Pages F, Berger A, Camus M, et al. Effector memory T cells, early metastasis, and survival in colorectal cancer. N Engl J Med 2005; 353:2654-66.
15. Galon J, Costes A, Sanchez-Cabo F, et al. Type, density, and location of immune cells within human colorectal tumors predict clinical outcome. Science 2006; 313: 1960-4.
16. Galon J, Fridman W H, Pages F. The adaptive immunologic microenvironment in colorectal cancer: a novel perspective. Cancer Res 2007; 67:1883-6.
17. Zhang L, Conejo-Garcia J R, Katsaros D, et al. Intratumoral T cells, recurrence, and survival in epithelial ovarian cancer. N Engl J Med 2003; 348:203-13.
18. Kalialis L V, Drzewiecki K T, Mohammadi M, Mehlsen A B, Klyver H. Spontaneous regression of metastases from malignant melanoma: a case report. Melanoma Res. 2008 August; 18(4):279-83.
19. Dudley M E, Wunderlich J R, Yang J C, et al. Adoptive cell transfer therapy following non-myeloablative but lymphodepleting chemotherapy for the treatment of patients with refractory metastatic melanoma. J Clin Oncol 2005; 23:2346-57.
20. Muranski P, Boni A, Wrzesinski C, Citrin D E, Rosenberg S A, Childs R, Restifo N P. Increased intensity lymphodepletion and adoptive immunotherapy—how far can we go? Nat Clin Pract Oncol. 2006 December; 3(12):668-81
21. Kmieciak M, Knutson K L, Dumur C I, Manjili M H. HER-2/neu antigen loss and relapse of mammary carcinoma are actively induced by T cell-mediated anti-tumor immune responses. Eur J Immunol 2007; 37:675-85.
22. Manjili M H, Arnouk H, Knutson K L, et al. Emergence of immune escape variant of mammary tumors that has distinct proteomic profile and a reduced ability to induce “danger signals”. Breast Cancer Res Treat. 2006; 96:233-41.
23. Guy C T, Webster M A, Schaller M, Parsons T J, Cardiff R D, Muller W J. Expression of the neu protooncogene in the mammary epithelium of transgenic mice induces metastatic disease. Proc Natl Acad Sci USA 1992; 89:10578-82.
24. Takeuchi N, Hiraoka S, Zhou X Y, et al. Anti-HER-2/neu immune responses are induced before the development of clinical tumors but declined following tumorigenesis in HER-2/neu transgenic mice. Cancer Res 2004; 64:7588-95.
25. Kmieciak M, Morales J K, Morales J, Grimes M, Manjili M H. Danger signal and nonself entity of tumor antigen are both required for eliciting effective immune responses against HER-2/neu positive mammary carcinoma: implications for vaccine design. Cancer Immunol Immunother 2008 (In press).
26. Knutson K L, Almand B, Dang Y, Disis M L. Neu antigen-negative variants can be generated after neu-specific antibody therapy in neu transgenic mice. Cancer Res 2004; 64:1146-51.
27. Wang E, Miller L, Ohnmacht G A, Liu E, Marincola F M. High fidelity mRNA amplification for gene profiling using cDNA microarrays. Nature Biotech 2000; 17:457-9.
28. Wang E. RNA amplification for successful gene profiling analysis. J Transl Med 2005; 3:28.
29. Jin P, Zhao Y, Ngalame Y, et al. Selection and validation of endogenous reference genes using a high throughput approach. BMC Genomics 2004; 5:55.
30. Rubinfeld B, Robbins P, el Gamil M, Albert I, Porfiri E, Polakis P. Stabilization of beta-catenin by genetic defects in melanoma cell lines. Science 1997; 275:1790-2.
31. Eisen M B, Spellman P T, Brown P O, Botstein D. Cluster analysis and display of genome-wide expression patterns. Proc Natl Acad Sci USA 1998; 95:14863-8.
32. Ross D T, Scherf U, Eisen M B, et al. Systematic variation in gene expression patterns in human cancer cell lines. Nature Genetics 2000; 24:227-35.
33. Wang E, Miller L D, Ohnmacht G A, et al. Prospective molecular profiling of subcutaneous melanoma metastases suggests classifiers of immune responsiveness. Cancer Res 2002; 62:3581-6.
34. Basil C F, Zhao Y, Zavaglia K, et al. Common cancer biomarkers. Cancer Res 2006; 66:2953-61.
35. Kaech S M, Hemby S, Kersh E, Ahmed R. Molecular and functional profiling of memory CD8 T cell differentiation. Cell 2002; 111:837-51.
36. Panelli M C, Stashower M, Slade H B, et al. Sequential gene profiling of basal cell carcinomas treated with Imiquimod in a placebo-controlled study defines the requirements for tissue rejection. Genome Biol 2006; 8:R8.
37. Mantovani A, Romero P, Palucka A K, Marincola F M. Tumor immunity: effector response to tumor and the influence of the microenvironment. Lancet 2007; in press.
38. Sinha P, Clements V K, Bunt S K, Albelda S M, Ostrand-Rosenberg S. Cross-talk between myeloid-derived suppressor cells and macrophages subverts tumor immunity toward a type 2 response. J Immunol 2007; 179:977-83.
39. Melani C, Chiodoni C, Formi G, Colombo M P. Myeloid cell expansion elicited by the progression of spontaneous mammary carcinomas in c-erbB-2 transgenic BALB/c mice suppresses immune reactivity. Blood 2003; 102:2138-45.
40. Wu K, Bi Y, Sun K, Wang C. IL-10-producing type 1 regulatory T cells and allergy. Cell Mol Immunol 2007; 4:269-75.
41. Biswas S K, Gangi L, Paul S, et al. A distinct and unique transcriptional programme expressed by tumor-associated macrophages: defective NF-{kappa}B and enhanced IRF-3/STAT1 activation. Blood 2006; 107:2112-22.
42. Fenner J E, Starr R, Cornish A L, et al. Suppressor of cytokine signaling 1 regulates the immune response to infection by a unique inhibition of type I interferon activity. Nat Immunol 2006; 7:33-9.
43. Mansell A, Smith R, Doyle S L, et al. Suppressor of cytokine signaling 1 negatively regulates Toll-like receptor signaling by mediating Mal degradation. Nat Immunol 2006; 7:148-55.
44. Qin F I, Wilson C A, Lee S J, Benveniste E N. IFN-beta-induced SOCS-1 negatively regulates CD40 gene expression in macrophages and microglia. FASEB J 2006; 20:985-7.
45. Li Y, Chu N, Rostami A, Zhang G X. Dendritic cells transduced with SOCS-3 exhibit a tolerogenic/DC2 phenotype that directs type 2 Th cell differentiation in vitro and in vivo. J Immunol 2006; 177:1679-88.
46. Fojtova M, Boudny V, Kovarik A, et al. Development of IFN-gamma resistance is associated with attenuation of SOCS genes induction and constitutive expression of SOCS 3 in melanoma cells. Br J Cancer 2007; 97:231-7.
47. Kroemer G, Hirsch F, Gonzalez-Garcia A, Martinez C. Differential involvement of Th1 and Th2 cytokines in autoimmune diseases. Autoimmunity 1996; 24:25-33.
48. Sasaki S, Amara R R, Yeow W S, Pitha P M, Robinson H L. Regulation of DNA-raised immune responses by cotransfected interferon regulatory factors. J Virol 2002; 76:6652-9.
49. Hunter C A. New IL-12-family members: IL-23 and IL-27, cytokines with divergent functions. Nat Rev Immunol 2005; 5:521-31.
50. Afzali B, Lombardi G, Lechler R I, Lord G M. The role of T helper 17 (Th17) and regulatory T cells (Treg) in human organ transplantation and autoimmune disease. Clin Exp Immunol 2007; 148:32-46.
51. Hao J S, Shan B E. Immune enhancement and anti-tumour activity of IL-23. Cancer Immunol Immunother 2006; 55:1426-31.
52. Shan B E, Hao J S, Li Q X, Tagawa M. Antitumor activity and immune enhancement of murine interleukin-23 expressed in murine colon carcinoma cells. Cell Mol Immunol 2006; 3:47-52.
53. Fulton A, Miller F, Weise A, Wei W Z. Prospects of controlling breast cancer metastasis by immune intervention. Breast Dis 2006; 26:115-27.

The complete contents of all references, patent applications and issued patents cited herein is hereby incorporated by reference.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for determining a probability of relapse of a patient with a tumor, comprising the steps of
obtaining a sample of said tumor;
measuring expression levels of immune system genes associated with said tumor;
determining, using said expression levels of said immune system genes, a pattern of expression of immune system genes associated with said tumor; and,
based on said pattern of expression, assigning said patient to a group selected from the group consisting of:
i) patients who are likely to experience a relapse; and
ii) patients who are unlikely to experience a relapse.

2. The method of claim 1, wherein said immune system genes include at least two genes listed in Tables 4 and 5.

3. The method of claim 2, wherein said immune system genes include at least one gene listed in Table 4 and at least one gene listed in Table 5.

4. The method of claim 2, wherein said immune system genes include all genes listed in Table 4 and Table 5.

5. The method of claim 1, wherein said immune system genes are from one or more pathways selected from the group consisting of B cell development, antigen presentation, graft vs host disease signaling, interferon signaling and primary immunodeficiency signaling.

6. The method of claim 1, wherein said pattern of expression of immune system genes shows upregulation of one or more genes listed in Table 4 and downregulation of one or more genes listed in Table 5, and said patient is assigned to said group of patients who are unlikely to experience a relapse.

7. The method of claim 1, wherein said pattern of expression of immune system genes shows upregulation of one or more genes listed in Table 5 and downregulation of one or more genes listed in Table 4, and said patient is assigned to said group of patients who are likely to experience a relapse.

8. The method of claim 1, wherein said tumor is a breast cancer tumor.

9. A method for developing a treatment protocol for a patient with a tumor, comprising the steps of
obtaining a sample of said tumor;
determining a pattern of expression of immune system genes in said tumor;
based on said pattern of expression of immune system genes, categorizing said patient as a patient that is likely to undergo a relapse or as a patient that is unlikely to undergo a relapse; and
developing a treatment protocol for said patient based on results obtained in said categorizing step.

10. The method of claim 9, wherein said immune system genes comprise genes listed in Table 4 and Table 5.

11. The method of claim 9, wherein said pattern of gene expression of immune system genes shows upregulation of one or more genes listed in Table 5 and downregulation of one or more genes listed in Table 4, said patient is categorized as likely to undergo a relapse, and said treatment protocol developed in said developing step is an aggressive treatment protocol.

12. The method of claim 9, wherein said pattern of gene expression of immune system genes shows upregulation of one or more genes listed in Table 4 and downregulation of one or more genes listed in Table 5, said patient is categorized as unlikely to undergo a relapse, and said treatment protocol developed in said developing step is a non-aggressive treatment protocol.

13. A system for determining a probability of relapse of a patient with a tumor, said system comprising
means for obtaining measurements of expression of immune system genes in tumors;
means for recognizing, using said measurements, patterns of gene expression, wherein said patterns of gene expression are correlated with said probability of relapse; and
means for assigning a probability of relapse to said patient with said tumor.

14. The system of claim 10, wherein said immune system genes comprise genes listed in Table 4 and Table 5.

15. A microarray chip for analyzing the likelihood of relapse of a patient with a tumor, comprising
primers specific for amplifying RNA corresponding to genes listed in-Table 4 and Table 5.
